(19) United States
(12) Patent Application Publication
(10) Pub. No.: US 2026/0275327 A1
(43) Pub. Date: Sep. 17, 2026

(54) UNIFORM MULTI-LAYER SILICA COATED POROUS/MESOPORUS MAGNETIC BEADS, METHODS OF MAKING AND USES THEREOF

(71) Applicant: MAGNOSTICS LIMITED, Monkstown (IE)

(72) Inventors: Gil LEE, Monkstown (IE); Ying WANG, Monkstown (IE); Peng LI, Monkstown (IE); Anne CHEVALIER, Monkstown (IE)

(21) Appl. No.: 19/670,363

(22) Filed: May 7, 2026

Related U.S. Application Data
(63) Continuation of application No. PCT/US2024/055006, filed on Nov. 7, 2024.
(60) Provisional application No. 63/596,839, filed on Nov. 7, 2023.

(51) Int. Cl. C12N 15/10; B01J 20/10; B01J 20/28; B01J 20/32

(52) U.S. Cl. CPC C12N 15/1013; B01J 20/103; B01J 20/28007; B01J 20/28009; B01J 20/28019; B01J 20/3204; B01J 20/3223; B01J 20/3236; B01J 20/3246; B01J 20/3293

(57) ABSTRACT

Methods for preparing of uniform spherical magnetic beads between 50 nm to 5 microns. This involves the formation of a uniform emulsion of hydrophobically coated nanoparticles dispensed in volatile organic solvent in an aqueous solution contains thickening agent and surfactant. Control of the chemical and physical parameters of emulsification enables adjustment of bead size. Methods for coating a silica layer on the spherical magnetic beads including: 1) a continuous silicate layer around the iron oxide core; and 2) a porous silica layer that has a defined pore size between 1 and 200 nm and thickness between 1 and 10,000 nm. The silica layer can be functionalized with chemical groups, including amine or carboxyl, or coated with a range of biochemical compounds, including peptides, proteins, or DNA, to facilitate separation and/or reaction. Kits for performing size selective separation and/or reaction including the extraction of specific size nucleic acids, proteins and macromolecules.

### CROSS REFERENCE TO RELATED APPLICATION

This application is a bypass continuation of International PCT Application no. PCT/US2024/055006, filed Nov. 7, 2024, which application claims priority benefit of U.S. Provisional Application No. 63/596,839, filed Nov. 7, 2023, the entire content of each of which application is hereby incorporated herein by reference.

### INTRODUCTION

Magnetic micro/nano particles/beads have been emerging in many physical, chemical, and biomedical applications. For example, particles with superparamagnetism where the particles show no net magnetization in the absence of magnetic field and strong magnetization under external magnetic field, have become more attractive, particularly in biomedical applications. The superparamagnetic feature makes this type of magnetic beads highly efficient and useful in a series of sample treatment, isolation, and measurements procedures.

To make superparamagnetic particles at room temperature, a typical technical route is to reduce the particles' dimensions down to a threshold dimension, at which the thermal fluctuation is balanced with magnetic energy of dipole of magnetic particles, therefore, the particles become superparamagnetic. For example, the most popular material for superparamagnetic particles is iron oxide. When smaller than 30 nanometers at room temperature, the iron oxide particles exhibit superparamagnetism.

Due to their biocompatibility, as well as a very wide and highly flexible capacity of being functionalized on particles surface, iron oxide particles have been approved as ideal materials for biomedical applications and have been widely and successfully used for labelling biological macromolecules and cells for separation, detection, drug delivery, diagnostic targeted imaging, and DNA and viral RNA extraction and detection.

### SUMMARY

The present application relates to methodology for producing and surface coating of submicrometer to micrometer-sized spherical magnetic beads, which are high in magnetic moment, highly uniform in dimensions using emulsion templated assembly of highly packed magnetic nanoparticles inside, and multiple layers silica, including mesoporous silica structure, are coated on surface using sol-gel process and micro emulsification. Such beads have high surface-to-volume ratio and can be surface functionalized with different surface chemicals with very active surface affinity. They are versatile for different applications, such as physical, chemical, biological magnetic separation, biological imaging, DNA sequencing, biosensing, drug delivery, clinic diagnosis, DNA/RNA extractions, etc.

Methods for preparing uniform spherical nano/microsized beads, including: 1) providing magnetic nanoparticles with size between 1 nm to 100 nm and assembled magnetic beads sized between 50 nm to 5 microns; 2) hydrophobically coated nanoparticles dispensed in highly volatile organic solvent (such as cyclohexane, pentane, or toluene) that is immiscible with aqueous solution; 3) Aqueous solution contains thickening agent with adjustable viscosity, such as Polyvinylpyrrolidone (PVP), dextran, Polyvinyl alcohol (PVA), Polyethylene glycol (PEG), and Gelatin, and surfactant; 4) a microfluidic pre-mixer that has T-shape and two fluid inlets, one junction, a fluid outlet; 5) a microfluidic pre-mixer that has Y-shape and two fluid inlets, one junction, a fluid outlet; 6) a microfluidic pre-mixer that has cross-shape and two fluid inlets, one junction, two fluid outlets; 7) producing emulsion in pre-mixer by dispersing flow of the hydrophobic solvent contains nanoparticles in a continuous flow of aqueous solution that is immiscible with solvent; 8) producing emulsion in pre-mixer by dispersing flow of aqueous solution in a continuous hydrophobic solvent flow contains nanoparticles; 9) providing uniform emulsion by dispersing emulsion from pre-mixer to a rotating shear device; 10) providing a temperature control in shear device to control or monitor the temperature and viscosity of emulsion; 11) providing adjustment of emulsion droplet size by adjusting the temperature and viscosity of emulsion temperature; 12) providing evaporation of solvent in emulsion droplet by heating evaporation or spray evaporation to form assembled micrometer or submicrometer sized bead.

In one embodiment, the magnetic beads are spherical and assembled with densely packed magnetic nanoparticles inside the beads. As results, the magnetic materials content is high, for example higher than 50% of total mass of a bead. Thus, one benefit of the present application is the ability of producing magnetic beads that have high and uniform magnetic moment for each bead and the beads are superparamagnetic as the superparamagnetic nanoparticles are encapsulated inside the beads.

Another aspect of the instant magnetic beads is spherical and assembled with densely packed magnetic nanoparticles outside the beads. As results, the magnetic materials content is high, for example higher than 50% of total mass of a bead. Thus, one benefit is the ability of producing magnetic beads that have high and uniform magnetic moment for each bead and the beads are superparamagnetic as the superparamagnetic nanoparticles are assembled outside the beads.

Another benefit of the present application is to produce the magnetic beads that are uniform in diameter and with spherical shape.

Another benefit of the present application is the ability to produce the uniform beads in high production yield by using mechanical shear device and pre-mixers.

Another benefit of the present application is the ability to produce the uniform beads with controlled and narrow diameter distribution. The variation of coefficient of diameter of such beads is preferably less than 40%. The diameter of beads can be adjusted by changing the size of emulsion droplets. There are several ways to implement that, for example, changing the rotation speed of rotor of shear device, changing the concentration of nanoparticle in disperse solution, changing physical dimension of gap between rotor and stator of shear device, changing the composition of the aqueous solution, changing the temperature of emulsion. In one embodiment, the uniform beads ranging from about 50 nm to about 5 microns in diameters.

Another benefit of present application is to produce magnetic beads in a fast way by using highly volatile and immiscible (with aqueous solution) organic solvent for dispersing magnetic nanoparticles. The highly volatile and immiscible (with aqueous solution) solvent, such as cyclohexane, pentane, or toluene, reduce the evaporation time for emulsion droplets to be assembled to the beads.

Another benefit of present application is to produce uniform beads in a large production yield by using pre-mixing devices to create first emulsion droplets and then the first emulsion droplets are proceeded by shear device in sequence. The pre-mixers reduce the production waste and increase the size uniformity and production yield.

Another benefit of the present application is to use biocompatible aqueous solution combined with immiscible organic in where the magnetic particles are dispersed to produce uniform emulsion droplets. The viscosity of aqueous fluid can be adjusted by the concentration of chemical composite in the solution. The aqueous solution may comprise Polyvinylpyrrolidone (PVP), or dextran, or Polyvinyl alcohol (PVA), or Polyethylene glycol (PEG), or Gelatin, and surfactant are preferably used. In one embodiment, beads 50 nm-5 micrometer in diameters, the preferable molecular weights and concentrations of chemicals for the aqueous solution are: PVP molecular weight (g/mol): 10000-1.3 million, aqueous concentration (w/w): 1-10%; Dextran molecular weight (g/mol): 20000-100000, aqueous concentration (w/w): 1-30%; PVA molecular weight (g/mol): 30000-200000, aqueous concentration (w/w): 0.5-5%; Gelatin molecular weight (g/mol): 20000-100000, aqueous concentration (w/w): 1-8%; PEG molecular weight (g/mol): 1000-10000, aqueous concentration (w/w): 1-30%.

Another benefit of the present application is using integrated temperature control and real-time monitor for producing uniform emulsion droplets in shear device emulsification. There is ability to control droplets uniformity, dimensions, and production yields by adjusting the emulsion temperature through integrated temperature control.

Another benefit of the present application is using centrifugation to select the magnetic beads with preferable sizes to obtain highly uniform magnetic bead.

The methods of coating muti-layer silica over magnetic beads therein, including: 1) producing silicate layers using sodium silicate in aqueous solution; 2) providing method to separate magnetic beads using different magnetic strength during the silica coating process; 3) producing silica layer based on the first silicate layer using Störbe method; 4) providing effects of silica coatings with and without ultrasonication; 5) providing different roughness of the second silica surface resulted by different coating conditions; 6) further providing a third corrugated silica layer in the mix of aqueous solution and immiscible (with aqueous solution) organic solvent (such as cyclohexane, toluene, pentane, chloroform); 7) providing morphologies of coated silica mesoporous surface resulted by different reaction conditions regarding heating temperatures, sonication powers, types of co-surfactant with different concentrations, etc.; 8) providing difference of the bead-size/thickness of silica layer resulted by different coating conditions.

In one embodiment, silica coating provides a homogenous and negatively charged outer layer over magnetic beads leading to a spherical formation of beads. Therefore, the silica coated magnetic beads are stable, chemical-resistant, mono-dispersed in the solution without aggregations and versatile to be functionalized with various surface functional groups. These functional groups include but not limited to amine, carboxyl, azide, biological macromolecules such as proteins including streptavidin, antibodies, enzymes, peptides, oligo-dT, nucleic acids and other affinity groups like crown ethers. Some of these groups, such as amine groups, can be directly attached to a silica surface using the well know silane chemistries. Many other groups can be further coated on silica surface indirectly via various crosslinker reactions, for example, PEG, carboxyl-amine interaction, EDC/NHS, NHS-amine, azide-DBCO, azide-crown ether, etc. Proteins and antibodies can be conjugated to crosslinkers through their primary amine groups whereas their affinity molecules can be attached specifically via molecular recognitions. Surfaces can be coated with polymers and functional biological macromolecules coated on top of them. These can be polymerized from the surface or graft to the surface. These two chemistries will produce different coverages of the mesoporous beads, i.e., total coverage vs. coating of the outer surface.

Another benefit of the present application is providing methodology to create mesoporous silica surface that has a large surface area (large surface-to-volume ratio). In this case, silica layers with high roughness, porosity, or highly corrugated 3D structures are created and used. Large accessible surface area produced by these mesoporous silica layer structures renders high absorption and high reaction rate for the beads for various applications, such as DNA sequencing, nucleic acids separation/extraction, nucleic acid size selection, molecule screening and purification, drug delivery, highly sensitive transducers for sensing, diagnostics, gene therapy, environmental contaminant clean-up, cell labelling/separation, and the like. These beads hold significant promise in the field of chromatography. Chromatography, a versatile chemical separation method, segregates mixtures by selectively distributing their components between a mobile phase (e.g., liquid or gas) and a stationary phase (e.g., solid or gel). This differential interaction results in distinct compounds moving at varying rates, effectively achieving separation. Magnetic chromatography employs magnetic particles as the stationary phase, that can be separated using magnetic field gradients. Modification of these magnetic particles with specific ligands enables preferential binding to target molecules, facilitating the purification and isolation of biomolecules like proteins, nucleic acids, and cells. This technique finds valuable applications in biotechnology and medicine for the extraction of specific compounds from complex mixtures. Moreover, mesoporous magnetic particles offer distinct advantages in chromatography due to their magnetic properties, large specific surface area, and size-tunable mesoporous structure, enabling efficient capture and separation of target molecules with varying sizes. They may be utilized in various methods, including but not limited to affinity chromatography, solid-phase extraction, ion exchange chromatography, hydrophobic interaction chromatography, displacement chromatography and size-exclusion chromatography, for the purification and isolation of biomolecules.

An example of the benefit of the mesoporous beads is the tunable pore size/morphology of the surface can be designed to select specific target species based on their dimensions or molecular weights. This creates a new application of magnetic bead separations, for instance, positive or negative separation of specific size species. For example, silica coated magnetic beads are widely used to select DNA/RNA sequences longer than 100 bp for detection sequencing based on the hydrogen bonding of nucleic acids with silica. Mesoporous silica beads with specific pore sizes offer a method to selectively capture small DNA/RNA fragments shorter than 150 bp (e.g., circulated tumor DNA), specific oligonucleotides (primers and nucleotides from PCR) and other contamination and small molecules that could affect the data-reading.

The present application contemplates magnetic porous beads for reaction, as nanoporous and mesoporous materials control the reaction, i.e., the pore size controls the reactions. The present inventors contemplate immobilizing enzymes and reactive centers in the pores to control rates of reaction for biocatalysis. For example, specific chemical groups and biomolecules will be immobilized on the porous beads to take advantage of their increased surface area and size selectivity. One application would be the removal of materials from bioreactors that inhibit reactions. For example, ammonia is known to be a major limiting factor in the production of monoclonal antibodies in large scale cell cultures. Highly specific removal of ammonia from these reactors can be achieved using porous magnetic beads that have been coated with crown ethers. These porous magnetic beads would be added to the fed-batch bioreactors, their crown ether groups would specifically bind ammonia, and high gradient magnetic separation would be used to remove the beads from the reactor. In this way, the ammonia concentrations can be controlled to avoid levels that negatively impact antibody production. Other applications of functional porous magnetic beads would include the removal of biomolecular groups from blood for biosensing or biomedical application. For example, specific size nucleic acids can be bond to the silica pores in the mesoporous layer. It is understood that antibodies and enzymes immobilized in the pores of these micro-sized magnetic beads will be protected from proteolysis and normal clearance mechanisms from the bloodstream. Advantages of these multicomplex separation techniques include increased yield and control of the speed of reaction and separation.

Methods for preparing uniform spherical nano/microsized magnetic beads, including: 1) providing magnetic nanoparticles with size between 1 nm to 100 nm and assembled magnetic beads sized between 50 nm to 5 microns; 2) hydrophobically coated nanoparticles dispensed in highly volatile organic solvent (such as cyclohexane, pentane, or toluene) that is immiscible with aqueous solution; 3) Aqueous solution contains thickening agent, such as Polyvinylpyrrolidone (PVP), dextran, Polyvinyl alcohol (PVA), Polyethylene glycol (PEG), and Gelatin, which adjustable viscosity and surfactant; 4) a fluidic pre-mixer that has T-shape and two fluid inlets, one junction, a fluid outlet; 5) a fluidic pre-mixer that has Y-shape and two fluid inlets, one junction, a fluid outlet; 6) a fluidic pre-mixer that has cross-shape and two fluid inlets, one junction, two fluid outlets; 7) producing emulsion in pre-mixer by dispersing flow of the hydrophobic solvent contains nanoparticles in a continuous flow of aqueous solution that is immiscible with solvent; 8) producing emulsion in pre-mixer by dispersing flow of aqueous solution in a continuous hydrophobic solvent flow contains nanoparticles; 9) providing uniform emulsion by dispersing emulsion from pre-mixer to a rotating shear device; 10) providing a temperature control in shear device to control or monitor the temperature and viscosity of emulsion; 11) providing adjustment of emulsion droplet size by adjusting the temperature and viscosity of emulsion temperature; 12) providing evaporation of solvent in emulsion droplet by heating evaporation or spray evaporation to form assembled micrometer or submicrometer sized bead. 12) providing magnetic separation for size selection by using magnets 13) providing magnetic separation by size selection by using centrifugation. Methods for surface coating multi-layer silica on spherical magnetic beads thereof with size between 50 nm to 5 microns, including methods of stepwise coating three different silica layers: 1) producing a surrounding silicate layers around the iron oxide core; 2) producing smooth bulbous silica layers; 3) producing corrugated/mesoporous silica layers that have high surface-to-volume ratio. The parameters for coating each layer can be adjusted to create customized beads with different silica layer size, different types of pores, different size/depth of the pores. Moreover the outer silica layer can be further functionalized with specific reactive groups (including amine or carboxyl) or coated with a range of biochemical compounds (including peptides, proteins, antibodies) to facilitate usage in specific applications.

Methods for preparing of uniform spherical magnetic beads between 50 nm to 5 microns. This involves the formation of a uniform emulsion of hydrophobically coated nanoparticles dispensed in volatile organic solvent in an aqueous solution contains thickening agent and surfactant. Control of the chemical and physical parameters of emulsification enables adjustment of bead size. Methods for coating a silica layer on the spherical magnetic beads including: 1) a continuous silicate layer around the iron oxide core; and 2) a porous silica layer that has a defined pore size between 1 and 200 nm and thickness between 1 and 10,000 nm. The silica layer can be functionalized with chemical groups, including amine or carboxyl, or coated with a range of biochemical compounds, including peptides, proteins, or DNA, to facilitate separation and/or reaction. Kits for performing size selective separation and/or reaction including the extraction of specific size nucleic acids, proteins and macromolecules.

### Magnetic Bead and Kits Containing the Same

In one aspect, a magnetic bead, comprising a continuous, porous silica layer surrounding a magnetic core, the magnetic core comprising nanoparticles of magnetic metal oxide or of magnetic metal alloy.

In this aspect, the continuous, porous silica layer is microporous, mesoporous, or microporous; and/or
the continuous, porous silica layer is functionalized with a functional group, such as amine, carboxylic groups, lysine, aldehyde, azide, peptide, proteins, nucleic acid (DNA, RNA), saccharide, polysaccharide, like including streptavidin, antibodies, enzymes, peptides, oligo-dT, nucleic acids and other affinity groups like crown ethers and/or a polymer layer is covalently bonded between the continuous, porous silica layer the functional group, the polymer layer, e.g., being, PEG; and or
the magnetic core comprising nanoparticles having a metallic oxide or magnetic metal alloy like an iron oxide (γ-Fe₂O₃ and Fe₃O₄) or ferrites (CoFe₂O₄ and MnO·6ZnO·4Fe₂O₄), alloys of FeCo and FePt, and magnetic cores comprising cobalt, manganese, or rare earth magnetic metal; and/or the nanoparticles exhibit superparamagnetism property; and/or
a continuous, porous silica layer that has a defined average pore size between about 1 and 200 nm or between about 2 and 100 nm or between about 3 and 50 nm; and/or
a continuous, porous silica layer that has a defined average thickness between about 1 and 10,000 nm or between about 5 and 1,000 nm or between about 10-50 nm or between about 20-40 or between about 25-30 nm; and/or
a continuous, nonporous silica layer between the continuous, porous silica layer and the magnetic core and/or the continuous, nonporous silica layer has a thickness less than 20, 15, 10 nm.

In any aspect, a kit comprises a compartment containing any magnetic bead described herein.

### Method Making

In one aspect, a method of making any magnetic bead described herein, comprising forming a continuous, porous silica layer on the magnetic beads.

In this aspect, the porous layer is formed by:
emulsifying a surfactant, a co-surfactant, water-immiscible solvent, a catalyst, silane, the coated beads in an aqueous solution to form a Windsor III phase microemulsion; and/or
in any aspect, the surfactant is anionic surfactant and non-ionic surfactant, such as CTAB, CPB, CTAC, SDS, or Tween; and/or
the co-surfactant is chosen from alkyl alcohols, such as hexanol, n-butanol, n-pentanol, 2-propanol, styrene/polystyrene, (di)ethyl ether, (6S)-6-methyloctan-1-ol, 1-heptanol, 2,6-dimethylheptan-1-ol, 2-butyl-1-octanol, 2-methylbutan-1-ol, 3,7,11-trimethyldodecan-1-ol, 3,7-dimethyl-3-octanol, 4-methylpentan-1-ol, ethanol, ethanol-d6, isoamylol, isobutanol, methanol, methanol-d1, pentanol; and/or
the water immiscible solvent is chosen from cyclohexane, methyphenyl ether, Octane, 1-Octadecene, decahydronaphthalene, Ethyl ether, Chlorobenzene, toluene, trialkylbenzenes, ethyl acetate, N,N-dimethyldecylamine, benzyl acetate, aldehyde; and/or
the catalyst is a base, e.g., urea or ammonium hydroxide, or an acid, e.g., acetate acid or hydrochloride acid concentrated sufficiently for deprotecting the silane; and/or
the silane is, e.g., TEOS and BTSE.

In any aspect, the continuous, porous silica layer is microporous, mesoporous, or macroporous.

In any aspect, the method, after forming the mesoporous magnetic beads, comprises removing surfactant and/or the removing utilizes a peroxide, such as hydrogen peroxide.

In any aspect, the method coats a layer of continuous porous silica over one or more magnetic beads, comprising: 1) optionally producing one or more silicate layers using ion-exchanged sodium silicate in aqueous solution; 2) separating magnetic beads using different magnetic strength during the silica coating process; keeping silicate coated magnetic beads in alkalic solution before next step; 3) producing one or more silane layers based on the first silicate; 4) producing a silane layer-coating, with optional sonication; 5) providing different roughness of the above mentioned silane surface resulted by different coating conditions; 6) producing a third corrugated silane layer in the mix of aqueous solution and immiscible (with aqueous solution) organic solvent (such as cyclohexane, toluene, pentane, chloroform); 7) providing morphologies of coated silica mesoporous surface resulted by different reaction conditions regarding heating temperatures, sonication powers, types of co-surfactant with different concentrations, types of catalysts, etc.; and 8) providing difference of the bead-size/thickness of silica layer resulted by different coating conditions; and/or the porous is microporous, mesoporous, or macroporous.

In any aspect, a method for producing one or more uniform spherical nano/microsized magnetic beads, comprising: 1) providing magnetic nanoparticles with size between about 1 nm to about 100 nm and assembled magnetic beads sized between about 50 nm to about 5 microns; 2) hydrophobically coating the nanoparticles dispensed in a highly volatile organic solvent that is immiscible with aqueous solution, wherein the aqueous solution comprises a thickening agent with adjustable viscosity and a surfactant; 3) producing an emulsion in a pre-mixer by dispersing flow of the hydrophobic solvent comprising the nanoparticles in a continuous flow of aqueous solution that is immiscible with the solvent; 4) producing emulsion in a pre-mixer by dispersing flow of aqueous solution in a continuous hydrophobic solvent flow comprising nanoparticles; 5) dispersing emulsion from pre-mixer to a rotating shear device, thereby providing uniform emulsion, temperature control, and viscosity control; 6) adjusting emulsion droplet size by adjusting the temperature and viscosity of emulsion temperature; and 7) evaporating solvent in emulsion droplet to form assembled micrometer or submicrometer sized bead.

In this aspect, the highly volatile organic solvent are selected from cyclohexane, pentane, or toluene; and/or
the thickening agent with adjustable viscosity is selected from Polyvinylpyrrolidone (PVP), dextran, Polyvinyl alcohol (PVA), Polyethylene glycol (PEG), and Gelatin; and/or
the evaporating solvent occurs by heating evaporation or spray evaporation; and/or one or more magnetic beads have uniform diameter and spherical shape; and/or
the magnetic beads have a uniform diameter ranging from about 50 nm to about 5 microns; and/or
the magnetic beads have a large mass of magnetic material, wherein the magnetic material comprises at least about 50% of total mass of the bead.

### Method Using

In one aspect, a method separates a target analyte in a sample, comprising binding the target analyte with any magnetic bead described herein, wherein the binding is in pores of the continuous, porous silica layer.

In any aspect, the separation is size exclusion, affinity separation, ion-exchange separation, reverse phase, hydrophobic separation, or bio-separation; and/or
the sample includes a matrix, such as tissue biopsies, formalin-fixed paraffin-embedded (FFPE) tissues, liquid biopsies (including blood, plasma, serum, saliva, urine, cerebrospinal fluid, amniotic fluid), cell lysates, cell culture supernatants, stool, sputum, buccal swabs, bone marrow, agricultural materials, plant tissues, water, soil and other environmental samples as well as microorganisms like bacteria, viruses, and fungi; and/or
the sample is modified by a modifying agent, such as buffering reagents such as Tris-HCl with various concentrations of chaotropic agents (such as guanidinium thiocyanate (GITC) or guanidine hydrochloride (GuHCl)), reducing agents (such as dithiothreitol (DTT) or β-mercaptoethanol), surfactant (such as Tween, triton X-100, SDS, etc.), salt (such as sodium chloride (NaCl), magnesium chloride (MgCl₂) etc.), chelating agents (such as ethylenediaminetetraacetic acid (EDTA)), enzyme (such as proteinase K) and optionally DNA/RNA carrier such as polyadenylic acid (Poly A) for RNA extraction or low-concentration DNA extraction and/or optionally washed after modification with a washing reagent; and/or
the target analyte is chosen from nucleic acids, such as nucleic acids, like all DNA, all RNA, cfDNA, ctDNA, cffDNA, RNA, messenger RNA (mRNA), microRNA (miRNA), long non-coding RNA (lncRNA), transfer RNA (tRNA) and/or ribosomal RNA (rDNA) and/or proteins, peptides, polysaccharides, small molecules, such biomarkers like CRP (C-Reactive Protein), Hemoglobin A1C, Insulin, RBC Magnesium, Testosterone, triglyceride/HDL Ratio, lipid panel, antibodies, prostate-specific antigen (PSA), Brain natriuretic peptide (BNP), HER2/neu, Vitamin D; and/or further detecting the separated target analyte and/or eluting the separated target analyte bound to the magnetic bead and thereafter detecting the eluted, separated target analyte.

In one aspect, provided is a method for producing one or more uniform spherical nano/microsized magnetic beads, comprising:
1) providing magnetic nanoparticles with size between about 1 nm to about 100 nm and assembled magnetic beads sized between about 50 nm to about 5 microns;
2) hydrophobically coating the nanoparticles dispensed in a highly volatile organic solvent that is immiscible with aqueous solution, wherein the aqueous solution comprises a thickening agent with adjustable viscosity and a surfactant;
3) producing an emulsion in a pre-mixer by dispersing flow of the hydrophobic solvent comprising the nanoparticles in a continuous flow of aqueous solution that is immiscible with the solvent;
4) producing emulsion in a pre-mixer by dispersing flow of aqueous solution in a continuous hydrophobic solvent flow comprising nanoparticles;
5) dispersing emulsion from pre-mixer to a rotating shear device, thereby providing uniform emulsion, temperature control, and viscosity control;
6) adjusting emulsion droplet size by adjusting the temperature and viscosity of emulsion temperature; and
7) evaporating solvent in emulsion droplet to form assembled micrometer or submicrometer sized bead.

In one embodiment, the highly volatile organic solvent is selected from cyclohexane, pentane, or toluene.

In one embodiment, the thickening agent with adjustable viscosity is selected from Polyvinylpyrrolidone (PVP), dextran, Polyvinyl alcohol (PVA), Polyethylene glycol (PEG), and Gelatin.

In one embodiment, the evaporating solvent occurs by heating evaporation or spray evaporation.

In one embodiment, the one or more magnetic beads have uniform diameter and spherical shape.

In one embodiment, the magnetic beads have a uniform diameter ranging from about 50 nm to about 5 microns.

In one embodiment, the magnetic beads have a large mass of magnetic material, wherein the magnetic material comprises at least about 50% of total mass of the bead.

In another aspect, provided is a method for coating a layer of continuous silica over one or more magnetic beads, comprising:
1) optionally producing one or more silicate layers using ion-exchanged sodium silicate in aqueous solution;
2) separating magnetic beads using different magnetic strength during the silica coating process; keeping silicate coated magnetic beads in alkalic solution before next step;
3) producing one or more silane layers based on the first silicate;
4) producing a silane layer-coating, with optional sonication
5) providing different roughness of the above-mentioned silane surface resulted by different coating conditions;
6) producing a third corrugated silane layer in the mix of aqueous solution and immiscible (with aqueous solution) organic solvent (such as cyclohexane, toluene, pentane, chloroform);
7) providing morphologies of coated silica mesoporous surface resulted by different reaction conditions regarding heating temperatures, sonication powers, types of co-surfactant with different concentrations, types of catalysts, etc.; and
8) providing difference of the bead-size/thickness of silica layer resulted by different coating conditions.

In one embodiment, the bead is microporous, mesoporous, or macroporous.

In one aspect, provided is a kit comprising microporous, mesoporous, or macroporous silica beads with specifically designed pore sizes, the appropriate buffers (adsorption, rinse, and desorption) and instructions for use.

In one aspect, provided is a method for affinity separation, comprising using any composition and/or methodology disclosed herein.

In one aspect, provided is a method for ion-exchange separation, comprising using any composition and/or methodology disclosed herein.

In one aspect, provided is a method for bio-separation, comprising using any compositions and/or methodology disclosed herein.

In one aspect, provided is a use of magnetic porous beads for controlling rate of a reaction, wherein the pore size controls the reaction. In one embodiment, the pores comprise an immobilized enzyme and reactive center.

In one aspect, provided is a use of a mesoporous material for controlling rate of a reaction.

In one aspect, provided is a mesoporous magnetic bead with a silica layer.

In one aspect, provided is a kit for extracting nucleic acids, wherein said kit comprises a mesoporous magnetic bead with a silica layer, wherein said bead has stronger binding capacities for nucleic acids than flat silica surfaces.

In one embodiment, nucleic acids comprise ssDNA, dsDNA, ssRNA, and dsRNA.

In one aspect, provided is a composition for extracting short single-stranded oligonucleotides, comprising a silica coated magnetic bead with pores of suitable diameter and depth (LP pores).

In one aspect, provided is a method for separating small nucleic acid fragments from a complex matrix, comprising using a porous magnetic bead with a silica layer to bind the nucleic acid fragments. In one embodiment, the nucleic acid fragments are ctDNA having a length of less than about 300 bp. In embodiment, the complex matrix comprises plasma or other biological fluids disclosed herein.

In one aspect, provided is a method for size-selective extraction of DNA, comprising using one or more carboxyl-coated mesoporous magnetic beads to bind the DNA. In one embodiment, the DNA ranges from about 25 bp to about 300 bp. In a further embodiment, the DNA size-selection can be well controlled by varying the mass/volume of beads/extraction buffer, and/or varying the among of PEG and NaCl in the extraction buffer, and/or varying the buffer content (such as a GITC based extraction), and/or bead type, and/or solvent (such as an alcohol, like methanol, ethanol, propanol, isopropanol, n-, sec-, or iso-butanol, etc.).

In one aspect, provided is a carboxyl-coated porous silica magnetic bead.

In one aspect, provided is a method for extracting cfDNA from biological fluid samples, comprising using one or more porous and/or mesoporous silica-coated beads to bind cfDNA.

In one aspect, provided is a method for extraction of nucleic acids from liquid biopsy, comprising using one or more porous and/or mesoporous silica-coated beads to separate cfDNA from biological fluid samples.

In one aspect, provided is a kit for extraction of nucleic acids, comprising one or more porous silica-coated beads.

In one aspect, provided is a method for increasing specific surface area of mesoporous silica-coated beads, comprising controlling pore size and pore structure of the silica layers. In one embodiment, beads having large pore (LP) and small pore (SP) silica layers both have significantly higher specific surface area compared to that of beads coated with smooth silica layer (SM). In one embodiment, the specific surface area increases by one or more orders of magnitude.

In one aspect, provided is a mesoporous silica-coated bead, wherein said bead comprises a surface functional group selected from amine, carboxyl, azide, proteins streptavidin, antibodies, enzymes, peptides, oligo-dT, nucleic acids, and crown ethers.

In one aspect, provided is a method for surface coating multi-layer silica on spherical magnetic beads, comprising stepwise coating three different silica layers: a) producing a surrounding silicate layers around the iron oxide core; and b) producing corrugated/mesoporous silica layers that have high surface-to-volume ratio.

In one embodiment, the parameters for coating each layer can be adjusted to create customized beads with different silica layer size, different types of pores, different size/depth of the pores.

In one embodiment, the outer silica layer can be further functionalized with specific reactive groups or coated with a range of biochemical compounds to facilitate usage in specific applications.

In one aspect, provided is a method for extracting a molecule from solution, comprising using a surface-coated multi-layer silica spherical magnetic bead, wherein said bead is coated with a functional group that interacts with said molecule and said molecule is smaller than the size of the pore of the bead. In one embodiment, the biochemical compounds are selected from peptides, proteins, and antibodies.

In one aspect, provided is a composition comprising SAv-modified LP beads.

In one aspect, provided is a composition comprising SAv-modified mesoporous beads with pore sizes of specific size.

In one aspect, provided is a method for separating biotin from solution, comprising using SAv-modified LP beads or SAv-modified LS beads.

In one aspect, provided is any methodology, use, device, composition, or kit disclosed herein.

### BRIEF DESCRIPTION OF THE DRAWINGS

FIG. 1 Assembly of magnetic beads by emulsification of two immiscible solutions, disperse phase that contains nanoparticles and continuous phase. The emulsions contains spherical droplets.

FIG. 2 Formation of magnetic beads from magnetic droplets. The emulsion droplets contain magnetic nanoparticles and continuous fluid, which is immiscible with initial solvent that disperses nanoparticles. The solvent in emulsion droplets will gradually evaporate thus the droplets will shrink in dimensions to for the solid magnetic beads, which assembled by numerous nanoparticles inside the beads.

FIG. 3 Emulsification technique by using a shear device for assembly of magnetic beads. A shear device consists of two mechanical concentric cylinders, a rotational rotor in the center and a static stator, with a narrow gap between them. A premixed emulsion, which can be formed by using various pre-mixing fittings, is introduced from the inlet. The dimension of emulsion is then reduced by shear gradient across the gap and flow out from the outlet to create uniform emulsion droplets. After evaporation, the droplets are assembled to beads with specific diameters.

FIG. 4A shear device is consisted of two concentric cylinders, a fixed stator and a rotational rotor, with a precise gap between them. A inlet and a outlet port on the stator are for premixed and processed emulsion to flow in and out. The roughness of inner surface of stator and rotor and gap between them are precised controlled. In this device, bearings, shaft seals, and motor are required to facilitate the rotation of shear device.

FIG. 5A A complete shear device that consists of a stator, a rotor that is concentric with stator, bearings and seals that support the rotational rotor and seal the emulsion inside the stator, an inlet that is connected with a pre-mixer for creating premixed emulsion, an outlet for out emulsion, an integrated temperature monitor connected with the outlet, and a temperature controlled fluid (TCF) manifold that controls the temperature of shear device and emulsion flows. FIG. 5B (b) is the cutaway view of a shear device. FIG. 5C is a transparent section view of a shear device. FIG. 5D is a transparent section view plus a cutaway (cut through lines a-b and a′-b′) from the inlet of a shear device.

FIG. 6A number of pre-mixeres are used to premix the emulsion before being introduced into the shear device for processing. For example, three different configurations are used for premixers, T-fitting, Y-fitting, and cross-fitting. A continuous fluid and one or two disperse fluid (only in cross-fitting) disperse fluid concurrent in the pre-mixer (or vice versa) then flow out from pre-mixer and then was introduced into the shear device from its inlet.

FIG. 7A temperature control fluid (TCF) manifold, in which fluid with adjustable temperature flow, is integrated into the shear device. The TCF is used to control the temperature of emulsion and shear device. An integrated temperature monitor (thermometer) is used to monitor the emulsion flow temperature in real-time. Left: transparent section view of shear device with TCF manifold. Right: cutaway view of TCF manifold.

FIG. 8A complete setup of a shear device for producing uniform magnetic emulsion droplets. The rotation of stator of shear device is controlled by a servo motor.

FIG. 9 Magnetic wash/separation by using magnets. (Left) Magnetic beads suspension (in the beaker) is captured by a magnet underneath the beaker. (Right) Magnetic beads suspension (in the beaker) is separated by a ring magnet.

FIG. 10 Transmission electron microscopic (TEM) image of magnetic nanoparticles.

FIGS. 11A and 11B Optical microscopic image of magnetic droplets out of shear device.

FIGS. 12A, 12B and 12C Each is a transmission electron microscopic (TEM) image of magnetic beads.

FIG. 13 Illustration of the stepwise coating of silica layers over the spherical magnetic core.

FIGS. 14A and 14B Each is a microscopic image of silicate and silica coated beads. FIG. 14A Beads coated using strong magnetic separation and without using sonication aggregate.

FIG. 14B Beads coated using weak magnetic separation and sonication remain separated.

FIGS. 15A and 15B Scanning electron microscopic (SEM) images of FIG. 15A smooth silica coated magnetic beads and FIG. 15B smooth silica coated magnetic beads with a higher surface roughness. Different roughness of the smooth silica layer can be achieved through varying the addition-rate of TEOS into the beads-mixture.

FIG. 16 Scanning electron microscopic (SEM) images of corrugated/mesoporous silica coated magnetic beads while coated without sonication power. [TEOS]=2 μL/mg beads, [beads]=8 mg/mL, urea as catalyst.

FIG. 17A SEM image of mesoporous small pore beads (SP). Insert: detailed SEM image of SP beads. The diameter of the pores are shown as about 5 nm. FIG. 17B TEM image of SP using accelerating voltage of 20 kV. FIG. 17C TEM image of SP using accelerating voltage of 200 kV. The thickness of the total silica layer is shown as about 50 nm with the pore depth of around 40 nm. FIG. 17D Optical microscopic image of finished SP beads.

FIGS. 18A, 18B, and 18C Scanning electron microscopic (SEM) images of mesoporous silica coated magnetic beads with either FIG. 18A 5% 1-pentanol added (mesoporous large pore beads LP) or FIG. 18B 1% 2-propanol added. FIG. 18C Transmission electron microscopic (TEM) image of mesoporous silica coated magnetic beads using accelerating voltage of 200 kV where TEOS was added into the reaction mixture in two times instead of dropwise.

FIG. 19A Illustration of an example of chemical modification to silica coated magnetic beads, with the functionalization of silica coated beads with amine, carboxyl and streptavidin (SAv). SAv coated magnetic beads, able to separate biotinylated molecules, were then incubated with biotin-fluorescein. FIG. 19B The biotin binding capacity (=slope of the curves) of SAv coated magnetic beads, with smooth or mesoporous silica layer, to biotin-fluorescein was evaluated. The binding capacity of mesoporous beads is proven to be double that of smooth beads. FIG. 19C Fluorescence intensities of unbound/free biotin-fluorescein molecules after incubation with smooth or mesoporous silica coated magnetic beads. Lower fluorescent intensity of the residue is shown by mesoporous beads than by smooth beads, indicating more absorption of dye molecules to mesoporous beads than to smooth beads.

FIGS. 20A-20D Gel electrophoresis images of single-stranded DNA (ssDNA) and double-stranded (dsDNA) extracted in various solutions/solvents by smooth (SM), small pore (SP) and large pore (LP) silica coated magnetic beads, respectively. Ultra low DNA ladder ranging from 10 to 300 bp was employed as size marker (well labelled as “M”) on a 4% agarose gel. Note that the ssDNA molecules migrate differently from dsDNA, thus ssDNA often show smear bands with altered positions. FIG. 20A DNA ladders were incubated with SM, SP and LP beads in deionized (DI) water at pH 5, with an expression of ssDNA formation; FIG. 20B DNA ladders were incubated with SM, SP and LP beads in 10 mM Tris-HCl at pH 5 performing as dsDNA; FIG. 20C DNA ladders were incubated with SM, SP and LP beads in 10 mM Tris-HCl at pH 5 with 3 M NaCl; FIG. 20D DNA ladders were incubated with SM, SP and LP beads in 80% ethanol with 200 mM MgCl₂. UV-Vis measurement were made for samples as of gel (FIGS. 20A, 20C and 20D) and (FIG. 20E for (a) (, i.e., FIG. 20A), FIG. 20F for (c) (, i.e., FIG. 20C), FIG. 20G for (d) (FIG. 20D) Extraction rate was evaluated for each assay, with P-values analysed using Kruskal-Wallis ANOVA test with a significance level of 0.05. The absorbance results of (b) (i.e., FIG. 20B) was not shown as the concentration of recovered DNA was too low for UV-Vis measurement.

FIGS. 21A and 21B Gel electrophoresis image of DNA extraction using silica coated smooth and mesoporous magnetic beads and lysis buffer containing guanidinium thiocyanate (GITC).

FIG. 21A DNA extractions were performed using smooth (SM) and small pore (SP) silica beads. DNA ladders (25-1000 bp) were spiked in fetal bovine serum (FBS) and extracted by SM and SP, respectively. GeneRuler Low Range DNA ladder (ranging from 25-700 bp) was used as size marker in well “M”. Initial DNA ladders (25-1000 bp) are shown in well 0. The gel image shows that mesoporous SP beads captured both longer and shorter fragments (below 100 bp) and SM beads could only extract the longer ones (over 100 bp). FIG. 21B DNA extraction in fetal bovine serum (FBS), with and without spike-in of nucleotide oligos, was performed using LP beads, respectively. Ultra Low Range DNA Ladders (10-300 bp) were used as Marker. Well “S” showed the result of recovered DNA without spike-in. The length between 150 and 200 bp indicated the recovery of endogenous cfDNA in FBS. Wells “25” and “50” show respectively the size profile of recovered DNA when 25 and 50 nt oligonucleotides were spiked-in. These results clearly prove that LP mesoporous beads can extract short single-stranded oligonucleotides.

FIG. 22 Comparison of the speed of separation of smooth (SM) silica coated magnetic beads (purple), small pore (SP) mesoporous beads (blue), and large pores (LP) mesoporous beads (green) by magnetic separator that used for deep-well 96 microplate separation. The separation process is monitored by measuring the transmittance of a green laser (550 nm wavelength) passing through the centre of a transparent cuvette, in which 2 mg beads were homogeneously dispersed (2 mL in purified water) initially. After the measurement started, the beads (in cuvette) were inserted into the separation position, which is aligned with the laser and sensor, of separator. The magnets on the separation attract the magnetic beads to the sidewall of the cuvette and the transmittance of the laser in the centre of cuvette increases. The separation of SM beads reached to over 90% within one minute and was 100% finished within three minutes. The separation of SP and LP mesoporous beads is slower compared to the SM beads, with 90% separations in about three minutes, and 100% completion of separation in less than 10 minutes, indicating rapid separation for all beads.

FIG. 23 Gel electrophoresis images of DNA ladders (size ranging from 10 to 2,500 bp) extracted with mesoporous silica coated magnetic beads that were incubated with hydrogen peroxide (H₂O₂) at room temperature for 0 to 15 min. Ultra low DNA ladder ranging from 10 to 300 bp was employed as size marker (well labelled as “M”). After the treatment with H₂O₂, both short (<100 bp) and long DNA fragments were captured, indicating that the soft templates of the surfactant used in the mesoporous silica coating (CTAB in this example) were efficiently removed hence allowing the mesopores to be accessible for DNA fragments.

FIGS. 24A and 24B Size selective DNA extraction using mesoporous SP beads. FIG. 24A (a) DNA extraction, with and without size-selection, was performed with SP beads. GeneRuler Low Range DNA ladder (ranging from 25-700 bp) was used as size marker in well “M”. The size profile of the initial spike-in DNA (25-1,000 bp) was shown in well 0, that of recovered fragments by DNA extraction without size-selection was shown in well 1. Recovered DNA fragments after size-selective extraction (25-300 bp) were expressed in well 2. Here, a 2-step extraction was performed. In the first step, long DNA fragments were removed by SM beads when a low volume of IPA was added to the lysis buffer. As a result, only the shorter DNA fragments (<300 bp) were extracted by SP beads in the second extraction step. FIG. 24B The threshold of the size-selection can be controlled varied via changing the composition of the lysis buffer including isopropanol in the first extraction step. Adding 80 μL isopropanol, instead of 50 μL, into the lysis buffer containing 6 M GITC, lowered the threshold of the DNA size-selection from 300 bp (well 300) to 200 bp (well 200). This kind of size selective extraction can be used to specifically extract small DNA, such as cell free DNA (cfDNA), circulating tumor DNA (ctDNA) in cancer studies, or cell-free fetal DNA (cffDNA) for prenatal testing.

FIG. 24C Results of the bioanalyzer analysis using high sensitivity DNA chip for amount and size distribution of the initial spiked-in DNA ladder (25-1,000 bp) in elution buffer at 20 ng/μL. This is a control showing the expected graphical profile for a 100% recovery for all DNA fragments. The numbers in the graphs corresponds to the size in base pairs of the DNA for each peak. FIG. 24D Results of the bioanalyzer analysis using high sensitivity DNA chip for amount and size distribution of the DNA recovered using the 1-step extraction procedure with SM beads, showing recovery of high proportion the longer DNA fragments (estimation of average 80% recovery for fragments 320-1,300 bp). The numbers in the graphs corresponds to the size in base pairs of the DNA for each peak. FIG. 24E Results of the bioanalyzer analysis using high sensitivity DNA chip for amount and size distribution of the DNA recovered using the 2-step extraction procedure with SM beads first, then SP beads. The numbers in the graphs corresponds to the size in base pairs of the DNA for each peak. The graphical profile reflects a good size selection for shorter DNA fragments (estimation of average 70% recovery for fragments of size ranging from 40 to 170 bp).

FIG. 25 Gel electrophoresis image of the size selection of longer and smaller DNA fragments using carboxyl-coated LS magnetic beads and PEG-based extraction buffers. GeneRuler Low Range DNA ladder (ranging from 25-700 bp) was used as size marker in well “M”. From the initial spiked-in DNA ladder (25-1,000 bp), well “A” shows the specific recovery of longer DNA fragments (>300 bp) using a 1-step procedure based on carboxyl-coated LS magnetic beads in a buffer containing 20% PEG-8000 and 2M NaCl. At the opposite, well “B”, from the same initial spiked-in DNA ladder (25-1,000 bp), shows the specific recovery of smaller DNA fragments (<300 bp) using a 2-step procedure in which carboxyl-coated LS magnetic beads in a buffer containing 20% PEG-8000 and 2M NaCl are first used to remove the larger DNA fragments. The supernatant from this reaction is then supplemented with carboxyl-coated LS magnetic beads in a buffer containing 30% PEG-8000 and 3M NaCl to specifically extract the smaller DNA fragments remaining in supernatant.

### DETAILED DESCRIPTION

As explained below, the present application relates to methodology for producing and surface coating of submicrometer to micrometer-sized spherical magnetic beads, which are high in magnetic moment, highly uniform in dimensions using emulsion templated assembly of highly packed magnetic nanoparticles inside, and multiple layers silica, including mesoporous silica structure, are coated on surface using sol-gel process and micro emulsification. The present inventors discovered that such beads have high surface-to-volume ratio and can be surface functionalized with different surface chemicals with very active surface affinity. They are versatile for different applications, such as physical, chemical, biological magnetic separation, biological imaging, DNA sequencing, biosensing, drug delivery, clinic diagnosis, DNA/RNA extractions, etc.

As the efficiency and worth of magnetic particles (or micro-sized beads) are closely associated with their magnetic moment and uniformity for the most applications, it is highly desirable to produce magnetic beads with high production yield and high uniformity not only in magnetic moment but also in dimensions.

Surface coatings for magnetic beads are essential for the most applications, as they provide functional possibilities. A silica outer layer enables magnetic beads to be resistant to harsh applicational environment, such as strong acidic/basic and different solvents. More than that, particles with silica coatings have wide applications in pharmaceutical, biomedicine, biotechnology, drug delivery, food, cosmetics, waste treatment, etc., due to their unique physiochemical properties. Particularly, silica-coated magnetic beads have been successfully used for large scale DNA/RNA extractions during the pandemic and have demonstrated their significance in biomedical and diagnostic area. Silica layer is also a good media for coating of multiple biochemical functional groups, such as amine, carboxylic groups, lysine, aldehyde, azide and proteins, via chemical- and bioconjugation, that makes silica coatings a popular alternative for various biomedical applications.

This present application provides methodology for coating three different silica layers with combination of smooth/bulbous silica layer and/or corrugated/mesoporous structures over magnetic beads. The coated beads have significantly high specific surface area. Smooth/bulbous silica layers can be synthesized through silica sol process with Störbe Method whilst corrugated/mesoporous silica layers are produced using microemulsion.

Reference will now be made in detail to the present embodiments, examples of which are illustrated in the accompanying drawings. Wherever possible, the same reference numbers will be used throughout the drawings to refer to the same or like parts.

The conjunction “and” or “or” can be used in the list of members, but the “at least one of” phrase is the controlling language. For example, at least one of A, B, and C is shorthand for A alone, B alone, C alone, A and B, B and C, A and C, or A and B and C.

Technical terminology in this description conforms to common usage in chemistry, physics, biology, material science, and the like.

Polyvinylpyrrolidone (PVP) is the U.S. Food and Drug Administration (FDA) approved chemicals for many uses. It is used in pharmaceutical, personal care, food additive, and medical applications. PVP polymers are available in different molecular weights. The aqueous or organic solvent solution of PVP with different molecular weights have a wide range of solubility and viscosity. That makes PVP an ideal nontoxic and biosafe thickening agent for multiple applications.

Dextran is a nontoxic polymer and widely used in pharmaceutical and medical applications. Soluble aqueous solution of Dextran with different molecular weights are used as emulsifying and thickening agents.

Polyvinyl alcohol (PVA) is a synthetic polymer which is biocompatible and widely used as thickening agent and emulsion stabilizer.

Polyethylene glycol (PEG) is a polyether compound and is widely used as additives in pharmaceuticals, cosmetics, and food. PEGs are available with wide range of molecular weight. The PEGs with different molecular weight have different physical properties. Typically, the PEG has a average molecular weight of less than 20,000 Da or 10,000 Da or 2,000 Da.

Gelatin is a hydrolyzed form of collagen and is widely used as a gelling agent in food, beverages, and pharmaceutical industries.

Sodium dodecyl sulfate (SDS) is an anionic surfactant used in many cleaning and hygiene products and as food additive as well.

Cyclohexane is a non-polar organic solvent with low solubility in water and high volatility. Pentane (or toluene) is a non-polar organic alkane solvent with extremely low solubility in water and high volatility.

Iron oxide nanoparticles (IONs) are nanoparticles exhibit superparamagnetism property when their dimensions are smaller than certain threshold size, for example, 30 nanometers. The IONs are considered biocompatible and are widely used in biomedical applications, such as, biosensing, bio-separation, bio-imaging, diagnosis, drug delivery, etc.

Sodium silicate is a soluble silicate which is often called “water glass”. Sodium silicate shows normally alkaline in pH. After ion-exchanged, the molecules are negatively charged and present an acidic with pH about 3-4.

Silane as used herein refers to chemical compounds which can form silica layers via sol-gel process. These compounds can be but not limited to tetraethyl or triethyl orthosilicate (TEOS), vinyltrimethoxysilane (VTMS), dimethyldiethoxysilane (DMDES), methyltrimethoxysilane (MTMS), 1,2-bis(triethoxysilyl) ethane 96 16068-37-4 (BTSE), isobutyltrimethoxysilane (IBTMS), octyltriethoxysilane (OTES), isocyanatopropyltriethoxysilane (ICPTES), and propyltrimethoxysilane (PTMS).

Porous or pore size refers to nano-, macro-, meso-, or microporous material, generally having a pore size of larger than 50 nm for macroporous, 2-50 nm for mesoporous and smaller than 2 nm for microporous material. Nanoporous materials generally have a pore size of 1-100 nm.

Tetraethyl orthosilicate (TEOS) is an ethyl silicate/silane and often used as crosslinker for silica polymer chemistry.

Cetyltrimethyl ammonium bromide (CTAB) is a cationic surfactant and has similar industrial applications as SDS. Other such surfactants include for example Cetylpyridinium bromide (CPB), Cetrimonium chloride (CTAC).

Hydrogen peroxide (H₂O₂) acts as an oxidizing agent and can be used to remove CTAB templates on the mesoporous silica beads formed during the synthesis.

Streptavidin (SAv) is a bacterially derived biotin-binding protein. It can specifically bind to biotin with a binding affinity of 10¹⁵M⁻¹. The protein has a molecule weight of 52 kDa with a globular subunit organization of 5 nm in size.

Biotin a water-soluble B vitamin (vitamin B7) found naturally in some foods and also in supplements which can specifically bind to streptavidin.

Fluorescein isothiocyanate (FITC) is a kind of fluorescein/fluorescent dye which is widely used for biological detecting applications such as flow cytometry and fluorescence microscopy.

Deoxyadenosine triphosphate (dATP) is a nucleotide used for DNA synthesis as a template of DNA polymerase.

Oligo(dT)18 Primer is a synthetic single-stranded 18-mer oligonucleotide with 5′- and 3′-hydroxyl ends. It is a kind of oligomer which is a small molecule often containing several repeating units.

Polyadenylic acid (Poly(A)) is a RNA with a chain length of 2100 to 10000 nucleotides and is often used as a carrier for quantitative precipitation of DNA and RNA.

Lambda DNA (λ-DNA) is a linear, double-stranded phage DNA containing 12 base pairs (bp) single-stranded complementary 5′-ends. It is derived from an Escherichia coli bacteriophage (Bacteriophage lambda cI857 Sam7) with a chain length of 48502 bp.

2-Amino-2-(hydroxymethyl)-1,3-propanediol (Tris base) is component of buffer solutions widely used in biochemistry and microbiology. The buffer is initially basic and can be titrated to any pH value typically with hydrocholoride acid (HCl) or acetic acid (HAc).

Guanidine thiocyanate (GITC) is a protein denaturant. In DNA extraction, it is used as a chaotropic agent. Other chaotropic agents include e.g. guanidinium chloride (GuHCl).

Dithiothreitol (DTT) is a reducing agent. Similar reducing agents include such as tris(2-carboxyethyl)phosphine (TCEP) and dithioerythritol (DTE).

Triton X-100 is a nonionic surfactant. Similar ones are e.g. polysorbates like the Tween series and alkyl polyglucosides like decyl glucoside.

Ethylenediaminetetraacetic acid (EDTA) is a chelating agent binding divalent metal ions.

DNA ladders are sets of synthesized DNA fragments with various yet well-defined lengths. They generally serve as the reference/molecular size marker for precise DNA sizing and approximate quantification with gel-electrophoresis.

Fetal bovine serum (FBS) is a widely used growth component in cell culture media. As a complex mixture, it is also often employed as a base medium in e.g. biological and drug researches where proteins, nucleic acid and other macromolecules are relevant. In some examples, it is used as the matrix for DNA extraction.

Liquid biopsies refer to a simple non-invasive sample of body fluid such as blood, urine, saliva, amniotic fluid, cerebrospinal fluid, and the like taken from an individual for analyzing specific biomarkers, including but not limited to circulating nucleic acids, that can permit diagnosis, guide personalized treatment, ensure treatment efficiency or evaluate recurrence of a disease. This provides an alternative to classic invasive tissue biopsy.

Cell-free DNA (cfDNA) refers to short circulating DNA fragments present in body fluids such as blood, urine, saliva, and the like. Analysis of cfDNA by molecular techniques (such as PCR and sequencing) is used as biomarker for a range of conditions.

Circulating tumor DNA (ctDNA) refers to short circulating DNA fragments derived from tumour cells and present in body fluids such as blood, urine, saliva, and the like. Analysis of ctDNA in body fluids such as blood, urine, saliva, and the lik by molecular techniques (such as PCR and sequencing) is a method that can be used as a non-invasive method to diagnose cancer, monitor cancer progression, detect specific mutations, and guide personalized treatment.

Cell-free fetal DNA (cffDNA) refers to short circulating DNA fragments present in the maternal blood and deriving from the foetus. Analysis of cffDNA by molecular techniques (such as PCR and sequencing) is a non-invasive prenatal screening method.

Mesoporous silica beads refer to mesoporous silica coated superparamagnetic beads.

### A. Emulsification Methodology

In one aspect, the present application provides emulsification methodology to assemble superparamagnetic nanoparticles, which are sized between about 1 nm to about 100 nm, hydrophobically coated magnetic nanoparticles suspended in organic solvents that are immiscible with aqueous solution, to form a large submicron or micron sized superparamagnetic spherical bead.

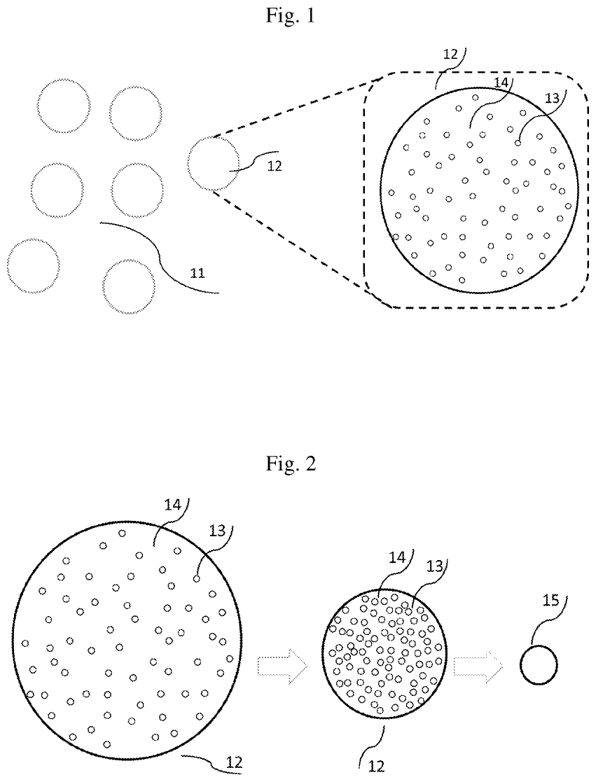

FIG. 1 illustrates certain aspects of a process to assemble submicron or micron sized beads. In some cases, the assembly of magnetic beads by emulsification of two or more immiscible solutions, disperse phase that contains nanoparticles and continuous phase. Here, the emulsion contains spherical droplets. The emulsion 11 created by emulsification method contains droplets 12 in which superparamagnetic nanoparticles 13 are encapsulated and clustered in organic solvent 14 (or in aqueous solution, e.g., if the bead with aqueous solution/polymer inside and nanoparticles assembled outside is produced). The droplet size ranges from about 0.2 micrometers to about 30 micrometers. During the evaporation process, the solvent in emulsion droplet evaporates and the droplet shrinks to a solid submicron or micron sized solid magnetic bead. 15, as illustrated in FIG. 2.

In another aspect, the application relates to assemble nanoparticles, which suspended in organic solvent, outside the emulsion droplet, while the polymer/aqueous solution is inside the emulsion droplet.

### B. Shear Device Emulsification and Pre-Mixers

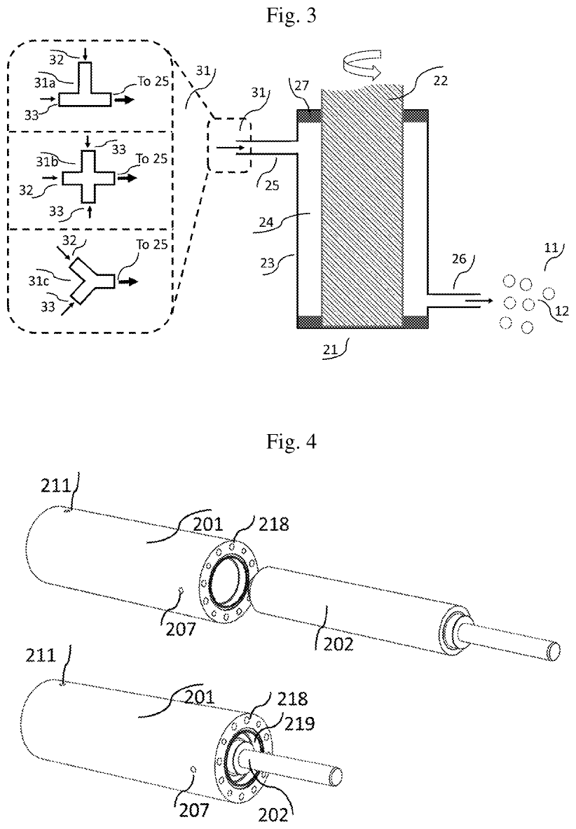

In one embodiment, emulsification methodology is based on shear device emulsification technique. FIG. 3 illustrates a shear device emulsification. For example, shear device 21 has two concentric mechanical cylinders, a rotational rotor 22 in the center and a fixed stator 23 around the rotor 21. The rotor and stator are assembled by rotor shaft bearings and seals 27 to maintain a gap 24 between them. There are one or two inlets 25 on stator and used to introduce the pretreated emulsion in the shear device. There is outlet 26 on the other end of stator to flow processed emulsion out of the shear device.

In another aspect, provided is a pre-mixing device 31 is used as an emulsification device to create the first set emulsion, which is subsequently introduced into the shear device through inlet of the shear device, as illustrated in FIG. 3.

There are generally three types of pre-mixers, as illustrated in the left-hand side in FIG. 3. For instance, the first type pre-mixer 31a has T-shape with two inlets 32 and 33, and an outlet that connected to inlet of shear device 25. Nanoparticles suspended in organic solution are introduced from inlet 32 and then concurrently mixed with aqueous solution that is introduced from inlet 33 inside the pre-mixer to form emulsion. The emulsion then flows to inlet 25 of shear device for secondary processing by shear device to produce uniform droplets. In another aspect, Nanoparticles suspended in organic solution are introduced from inlet 33 and then concurrently mixed with aqueous solution that is introduced from inlet 32 inside the pre-mixer to form emulsion. The emulsion then flows to inlet 25 of shear device for secondary processing by shear device to produce uniform droplets.

A second type pre-mixer 31b has cross-shape with three inlets 32 and two 33, and an outlet that connected to inlet of shear device 25. Nanoparticles suspended in organic solution are introduced from inlet 32 and then concurrent and mix with aqueous solution that is introduced from another two inlets 33 inside the pre-mixer to form emulsion. The emulsion then flows to inlet 25 of shear device for secondary processing by shear device to produce uniform droplets. In another preferable aspect, Nanoparticles suspended in organic solution are introduced from two inlets 33 and then concurrent and mix with aqueous solution that is introduced from inlet 33 inside the pre-mixer to form emulsion. The emulsion then flows to inlet 25 of shear device for secondary processing by shear device to produce uniform droplets.

A third type pre-mixer 31c has Y-shape with two symmetric inlets 32 and 33, and an outlet that connected to inlet of shear device 25. Nanoparticles suspended in organic solution are introduced from inlet 32 and then concurrently mixed with aqueous solution that is introduced from inlet 33 inside the pre-mixer to form emulsion. The emulsion then flows to inlet 25 of shear device for secondary processing by shear device to produce uniform droplets.

In one embodiment, the shear device is a rigid mechanical device with two concentric cylinders, as illustrated in FIG. 4. The rotational cylinder is the rotor 202 of shear device and mixed cylinder is the stator 201 of shear device. Between these assembled concentric cylinders, a gap is maintained. At least one inlet port 207 and one outlet port 211, which is in a distance from the inlet port, on the stator to introduce the premixed emulsion in and out. The rotational rotor creates a shear gradient on the emulsion in the gap to create uniform emulsion droplets with preferable sizes that related to the dimension of gap and rotating rate. Fluid channels 218 (optionally temperature controlled) encircle a bearing housing.

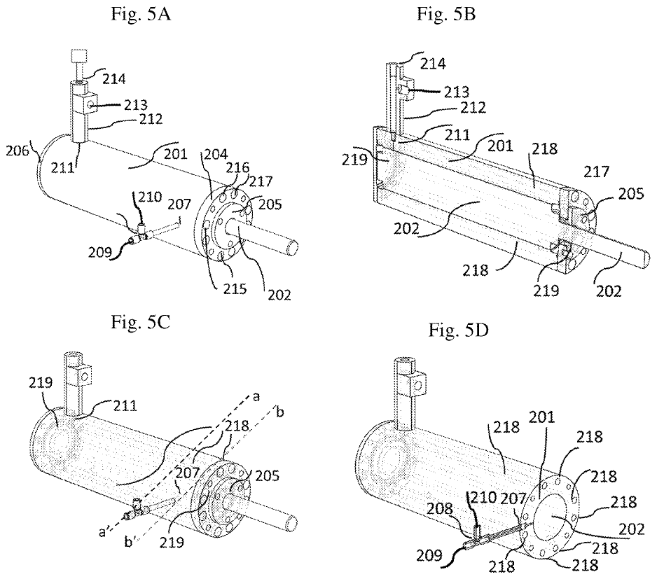

FIGS. 5A-5D illustrate the structure of a shear device. It has a mechanical rotor 202 that is controlled by a motor 103 through a stator 201 with at least one inlet port 207 and one outlet port 211. The pressure tight rotor-stator system is formed by a bearing housing 219, front seal cap 204, shaft seal housing 205 and end seal cap 206. This pre-mixer 208 is connected to inlet port of shear device and has at least one inlet and one outlet port, and is used to mix the organic mixture of magnetic nanoparticles with an aqueous solution. The components of the inlet port for the emulsion are first flow inlet 209, second flow inlet 210, outlet housing 212, and flow outlet 213. The temperature of the system is monitored with a thermometer/thermal couple 214 and controlled with the temperature control fluid (TCF) manifold 215, TCF inlet 216, TCF outlet 217 and TCF channels 218.

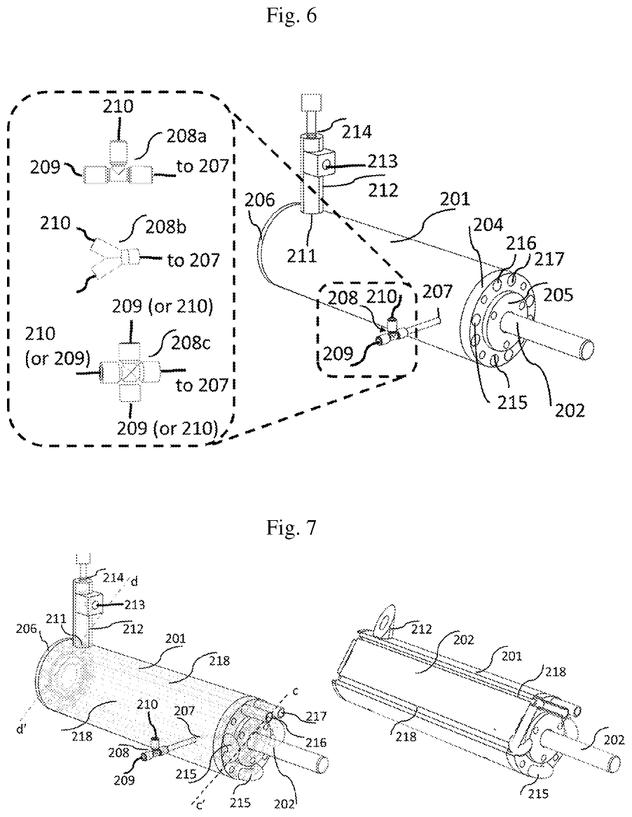

FIG. 6 illustrate the structure of three types of pre-mixers used for generating first set of emulsion that is introduced into the shear device. Another aspect relates to a temperature control fluid (TCF) manifold and monitor that are integrated on shear device to directly control and monitor the temperature of shear device and emulsion in real-time during processing of uniform emulsion droplets. As illustrated in FIG. 7, a TCF manifold 215 is a serials of fluid channels 218 (optionally temperature controlled) embedded inside the wall of stator of shear device. A close-loop or open loop of TCF is circulated through the TCF channels. The temperature of TCF is controlled by an external chiller or heater through port inlet 216 and outlet 217. As a result, the temperature of emulsion inside the gap (between rotor and stator) of shear device is controlled and adjusted by TCF.

In another aspect, the TCF channels are embedded inside the rotor of shear device to allow control of temperature of emulsion inside the shear device.

An integrated thermometer 214 is directly contact with emulsion inside the shear device to monitor the temperature of emulsion in real-time. The thermometer housing 212 is directly connected to the outlet (or inlet) of shear device. The thermometer housing has one or two ports that directly contact with emulsion to allow direct contact measurement of emulsion temperature by installed thermometer. Another port 213 on thermometer housing allows the same emulsion flow out of the shear device.

Combining TCF and thermometer, the temperature of emulsion inside the shear device can be controlled and monitor in real-time.

In an aspect, the temperature of emulsion inside the shear device is adjusted to produce uniform emulsion droplets. The viscosity of emulsion is adjusted by emulsion temperature. For example, the highest and the lowest emulsion temperature is not higher than boiling temperature and lower than freezing temperature of organic solvent and aqueous solution, respectively. A higher emulsion temperature has been used to produce large organic droplets/beads and a lower temperature is used for producing small droplets/beads.

### C. Processing to Select Desired Magnetic Beads

In an aspect, the obtained magnetic beads suspension from shear device is further processed by using a centrifugation procedure to select sized magnetic beads.

The centrifugation procedure is used to remove the magnetic beads that are not in the range of given sizes. For instance, magnetic beads are in the range of about 50 nm to about 5 microns. In a centrifugation procedure, the magnetic beads suspension (in aqueous solution with surfactant) is dispensed into a centrifuge bottle. Generally, the aqueous solution directly from shear device (after evaporation) is used, for example, aqueous solution with mixture of PVP and SDS. The sonication and shaking mixing are used to homogenously dispense the magnetic beads inside the centrifuge bottle. A 90-degree swinging-out centrifuge or a fixed-angle centrifuge can be used for centrifugation of the magnetic beads in the centrifuge bottle. The centrifuge time and relative centrifugal force are set based on type of centrifuge and desired sizes of magnetic beads. After centrifugation, the magnetic beads are settled on the bottom of centrifuge bottle and supernatant (solution with smaller and unwanted magnetic beads) are removed out of the centrifuge bottle. The supernatant can be removed by directly pouring out of the bottle or sucking out by using a suction apparatus.

The magnetic beads can be dispensed into an aqueous solution with density or viscosity gradient distributed vertically to perform differential centrifugation. For example, different concentrations of sucrose solution are used for such an aqueous solution with density or viscosity gradient.

The above centrifugation procedures can be repeated multiple times to increase the uniformity of selected magnetic beads.

In one embodiment, the magnetic beads are further processed by using magnetic wash—a procedure to select desired sized magnetic beads using magnets. In this procedure, magnetic beads in aqueous solution with surfactant (such as PVP SDS solution) is dispensed into a container (such as beaker) and homogenised by using sonication and mixing. And then, as illustrated in FIG. 9, magnetic beads suspension in container 301 are directly put onto a magnet 302 that has orientation of magnetization upwards as shown in FIG. 9. After certain duration (separation duration), the desired sized magnetic beads are captured on the bottom of the container and the supernatant (solution with smaller and unwanted magnetic beads) are removed out of the container by directly pouring or sucking out by a suction apparatus.

As shown in FIG. 9, a ring shape magnet 303, which has axial orientation of magnetization is used for magnetic wash. Here, the desired sized magnetic beads are captured on the side wall of container after a separation duration.

The separation duration depends on the type, dimension, and properties of magnet used. The separation duration also depends on the desired size, volume, and concentration of magnetic beads. The separation duration is also related to viscosity of magnetic beads solution.

A magnetic wash procedure can be repeated multiple times to increase the uniformity of selected magnetic beads.

### D. Silica Coatings on Magnetic Beads

The present application relates to multi-layer silica coatings on magnetic beads therein. In this regard, silica layers with various morphology and dimensions are fully covered on the outer surface of magnetic beads.

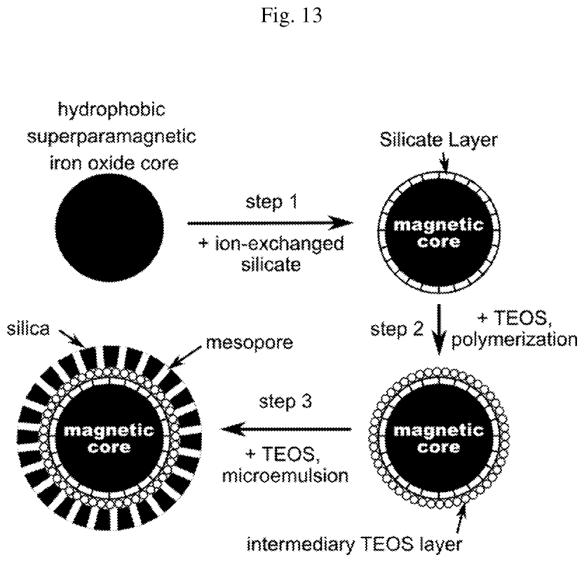

In one embodiment, methodology for coating silica layers on magnetic beads is illustrated in FIG. 13. First, a silicate layer is assembled on the magnetic beads surface as shown in FIG. 13, step 1. Prior to this step, sodium silicate is first ion-exchanged and then titrated with alkali to pH>7. The coating is performed in hydrophilic/aqueous solution. During that process, van der Waals and Coulomb energy lead to a tight surrounding however reversible bond of silicate to magnetic beads. Considering the binding competition between silicate and SDS, the concentration of SDS is usually reduced to lower than its critical micelle concentration (CMC) which is around 0.2% w/v at room temperature.

To produce monodispersed and non-aggregated magnetic beads with dense and homogenous distribution of silicate around them, several measures are presented: (a) suitable ultrasonication is used for preventing the aggregation of the magnetic beads, (b) pH value higher than 7 (for instance pH 8-11) prevents aggregation of magnetic beads in hydrophilic/aqueous solution, (c) A large number of free cations should be avoided, as cations shield the repulsive effect of charged beads and causes aggregation of beads and (d) The magnetic wash that involved in separation of magnetic beads using magnets should be performed with relative weak strength. In another aspect, a second silica layer is coated over beads using Störbe Method. An alkaline pH helps to keep beads separated and accelerate the reaction acting as catalyst. The surface roughness and thickness of silica layers are respectively defined by the speed and quantity of silane such as TEOS added to the bead's solution. Ultrasonication with various power (depending on the volume of the reaction and concentration of beads) may be applied during the coating to achieve the mono-dispersity and non-aggregation of beads. The silica layered coated in this step is a necessary as a media layer to receive the mesoporous silica layers and keeps the magnetic beads to be stable during the coating process. The thickness of this layer can be adjusted by changing the added amount of silane.

### E. Formation of Mesoporous Layers

The formation of mesoporous silica is a subject of long-standing interest with the early seminal work done in the 1990's at Mobile Research and Development (C. T. Kresge, M. E. Leonowicz, W. J. Roth, J. C. Vartuli, J. S. Beck, Nature 1992, 359, 710). These mesoporous materials are formed through templated synthesis in O/W emulsions and have a regular arrangement of cylindrical mesopores. They are characterized by an independently adjustable pore diameter (2 nm to 6.5 nm), a sharp pore distribution, and large surface area and pore volume. These materials have been used as a support for catalysts but have also been used for superior size exclusion separations (Cedric Boissire, Monika Kümmel, Michel Persin, Andre Larbot, and Eric Prouzet, Adv. Funct. Mater. 2001, 11, No. 2, 129-135).

To achieve a defined pore diameter, surfactants are used that form micelles in the synthesis solution. These micelles form templates that help build up the mesoporous framework. For MCM-41, cetyltrimethylammonium bromide (CTAB) has been used although polyethylene oxide-polystyrene and polyethyleneoxide-polyisoprene copolymers have also been used. The size of the pore can be changed by modified the quaternary ammonium surfactant compound or co-surfactants (J. S. Beck, J. C. Vartuli, W. J. Roth, M. E. Leonowicz, C. T. Kresge, K. D. Schmitt, C. T.-W. Chu, D. H. Olson, E. W. Sheppard, S. B. McCullen, J. B. Higgins, J. L. Schlenker, J. Am. Chem. Soc. 1992, 114, 10 834.). The silica mesoporous materials have often formed using the Strober reaction although it appears that nanometre scale sodium silicates can also be used.

Here, in the present application and in one embodiment, the pores are oriented normally to the surface of the silica bead surface so that the analyte can enter the pores in a rapid manner. This has been achieved by synthesizing a mesoporous silica layer using a Winsor III phase microemulsion. To reach this specific phase, the volume ratio of water to water-immiscible solvent is ideally about 1:2 to 1:1. Surfactant is applied to mix solvent to create the template for the growth of the silica structures with high surface-to-volume ratio. A co-surfactant is used to adjust the surface morphology of structure; thus the surface-to-volume ratio of the layer is adjusted accordingly. A wide range of water-immiscible solvents can be used, such as, cyclohexane, methyphenyl ether, Octane, 1-Octadecene, decahydronaphthalene, Ethyl ether, Chlorobenzene, toluene, trialkylbenzenes, ethyl acetate, N,N-dimethyldecylamine, benzyl acetate, aldehyde. Co-surfactants, normally but not limited to alkyl alcohols, such as hexanol, n-butanol, n-pentanol, 2-propanol, styrene/polystyrene, (di)ethyl ether, (6S)-6-methyloctan-1-ol, 1-heptanol, 2,6-dimethylheptan-1-ol, 2-butyl-1-octanol, 2-methylbutan-1-ol, 3,7,11-trimethyldodecan-1-ol, 3,7-dimethyl-3-octanol, 4-methylpentan-1-ol, ethanol, ethanol-d6, isoamylol, isobutanol, methanol, methanol-d1, pentanol, can be used to adjust Winsor III phase microemulsion to produce different morphologies of the mesoporous silica layer. Various surfactant, including anionic surfactant and non-ionic surfactant, such as CTAB, CPB, CTAC, SDS, Tween, can be (but not limited) used for the process. Various silanes, such as TEOS and BTSE can be (but not limited) used for the process. Various catalyst, such as urea, ammonium hydroxide, acetate acid, hydrochloride acid can be (but not limited) used in the process.

When the system remains in macroemulsion, bicontinuous microemulsion (i.e., interdispersed oil-phase and water-phase nanodomains separated by flexible surfactant monolayers) is well dispersed, and thus homogenous surface structures of silica layer can be formed. With different amount and types of solvents, co- and main surfactants, catalysts and silanes, adjustable morphology of silica structure, such as pore size, density of silica structure, dimensions of corrugated layers, etc., can be achieved. A suitable ultrasonication can be used to prevent the magnetic beads from aggregating.

### F. Kits

As explained throughout, the present application provides magnetic beads having mesoporous silica surface that have large specific surface area (large surface-to-volume ratio). Large surface area produced by these mesoporous silica layer structures renders high absorption and high reaction rate for the beads for various applications, such as DNA sequencing, nucleic acids separation, molecule screening and purification, drug delivery, highly sensitive transducers for sensing, etc. More than that, due to large absorption produced by mesoporous silica, the amount of beads needed in the reaction is significantly reduced, thus minimizing costs.

The present application contemplates one or more kits comprising magnetic beads, alone or in combination with other compositions, may be provided in the form of a kit. For example, components may be provided in a kit for physical, chemical, biological magnetic separation, biological imaging, DNA sequencing, biosensing, drug delivery, clinic diagnosis, DNA/RNA extractions, next generation sequencing (NGS), molecule purification, etc. Kits may further comprise appropriate controls and/or detection reagents.

Kits components will vary depending on the specific application, which determines surface morphologies and dimensions of the silica layers to gain advantages for a given application. For instance, for applications in harsh environment, such as strongly acidic solutions, the relatively thick silica layers can be coated on the magnetic beads; for applications requiring rapid separation, smooth thinner silica layers are preferred; for applications that require large surface interaction or absorption, the silica layers with large surface area, such as mesoporous or corrugated silica layers for magnetic beads; for beads with double silica layers, a very thin silica layer underneath the corrugated layer may work.

For example, a kit may consider tunable pore size/morphology of the silica surface for screening specific targets based on their difference in dimensions or molecular weights. That widens their applications in, for instance, next generation sequencing (NGS) and molecule purification, etc., for which the present application contemplates a kit and instructions. For example, the typical commercial silica beads can be used to select the sequence library longer than 100 bp. Instead, a kit with mesoporous silica beads with specifically designed pore sizes offer a method to selectively remove unwanted small DNA/RNA fragments shorter than 150 bp (e.g., adapter dimers), template residues (primers and nucleotides from PCR) and other contaminations and small molecules that could affect the data-reading.

For example, kits for nucleic acids (as a whole or for specific subtype of nucleic acids) might comprise mesoporous silica beads, with specifically designed pore sizes, and of various reagents (including but not limited to extraction, washing and/or elution reagents) for extraction of nucleic acids. The nucleic acid extracted might be of various types, including but not limited all nucleic acids, all DNA, all RNA, cfDNA, ctDNA, cffDNA, RNA, messenger RNA (mRNA), microRNA (miRNA), long non-coding RNA (lncRNA), transfer RNA (tRNA) and/or ribosomal RNA (rDNA). The matrix for the extractions is highly versatile and can include a wide range of sources such as tissue biopsies, formalin-fixed paraffin-embedded (FFPE) tissues, liquid biopsies (including blood, plasma, serum, saliva, urine, cerebrospinal fluid, amniotic fluid . . . ), cell lysates, cell culture supernatants, stool, sputum, buccal swabs, bone marrow, agricultural materials, plant tissues, water, soil and other environmental samples as well as microorganisms like bacteria, viruses, and fungi. In some cases, mesoporous silica beads might be functionalized with specific reactive groups, such as carboxyl, lysine or Oligo (dT). Extraction reagents may contain but are not limited to buffering reagents such as Tris-HCl with various concentrations of chaotropic agents (such as guanidinium thiocyanate (GITC) or guanidine hydrochloride (GuHCl)), reducing agents (such as dithiothreitol (DTT) or β-mercaptoethanol), surfactant (such as Tween, triton X-100, SDS, etc.), salt (such as sodium chloride (NaCl), magnesium chloride (MgCl₂) etc.), chelating agents (such as ethylenediaminetetraacetic acid (EDTA)), enzyme (such as proteinase K) and optionally DNA/RNA carrier such as polyadenylic acid (Poly A) for RNA extraction or low-concentration DNA extraction. Washing reagent(s) may contain but are not limited to ethanol, isopropanol, guanidine salts (such as GITC or GuHCl), buffering agent (such as Tris-EDTA buffer or Tris-HCl buffer), salts (such as NaCl, MgCl₂, etc.), detergents (such as Tween, triton X-100, SDS, etc.). Depending on the kit, one or multiple composition of lysis buffer(s) and of washing buffer(s) may be included. Elution reagents can be but not limited to Tris-EDTA buffer, Tris-HCl buffer, purified/nuclease-free water with pH values in the range of 7 to 10.

In some instances, the kit will also contain some specific pre-treatment reagents, such as proteinase K and SDS in the example of high-yield extraction of cfDNA from plasma, especially when the blood is collected in specialized tubes that stabilize cfDNA. The kit components may either be provided in their ready-to-use format or may require preparation by the end-user, including the addition of specific reagents such as ethanol or isopropanol. Solvents can contain but not limited to di-ionized/purified/nuclease-free water, isopropanol and ethanol. In some instance, additional steps might be included to improve the yield and the rate of extraction. This can be done, for example, through increasing the hydrophobicity of targeted nucleic acids or by inducing macromolecular crowding agents (with for example the use of polyethylene glycol (PEG) along with specific buffer containing salts). These kits may be sold as stand-alone products or as part of an integrated system. In the latter case, the extraction kit may be paired with additional components to facilitate nucleic acid analysis using various biomolecular techniques. This may include, but is not limited to, reagents for size selection of specific size nucleic acids, reverse transcription (RT) for cDNA synthesis, real-time PCR, digital PCR (dPCR), droplet digital PCR (ddPCR), sanger sequencing, microarray analysis, RNA sequencing, bisulfite sequencing, next-generation sequencing (NGS), etc.

In another example, a kit for size selective extraction of nucleic acids (as a whole or for specific subtype of nucleic acids) might comprise mesoporous silica beads, with specifically designed pore sizes, and various reagents (including but not limited to extraction, washing and/or elution reagents) for extraction of specific size nucleic acids. In such case, the mesoporous layer, the reagents and the procedure are optimized to selectively isolate specific size nucleic acids. The size selection might be from specific size nucleic acid binding into the pores, from the increased surface area from the mesoporous coating, and/or from the use of specific reagents and or steps in the procedure. These specific size nucleic acids might be of a wide range of type, including but not limited to cfDNA, ctDNA, cffDNA, miRNA and lncRNA. For example, the kit compounds might be optimized for DNA fragments shorter than 400 bp for extraction of cfDNA from liquid biopsies. The matrix for the extractions is highly versatile and can include a wide range of sources such as tissue biopsies, formalin-fixed paraffin-embedded (FFPE) tissues, liquid biopsies (including blood, plasma, serum, saliva, urine, cerebrospinal fluid, amniotic fluid . . . ), cell lysates, cell culture supernatants, stool, sputum, buccal swabs, bone marrow, agricultural materials, plant tissues, water, soil and other environmental samples as well as microorganisms like bacteria, viruses, and fungi. The kit reagents might be similar to the previous example, but the ratio for each component might be adjusted to allow for specific size selection. Moreover, additional steps in the procedure might be introduced, including but not limited to the use of different types of magnetic beads with specific properties and/or of specific buffer(s) optimized for size selection. Depending on the targets of the extraction, mesoporous silica beads can optionally be functionalized with carboxyl groups. In that case, additional reagents containing a mixture of PEG (with various molecular weight, including PEG8000) and salts (such as sodium chloride (NaCl)) with might be used for improving the size selectivity. Such kits may be sold as stand-alone products or as part of an integrated system. In the latter case, the size selective extraction kit may be paired with additional components to facilitate nucleic acid analysis using various biomolecular techniques. This may include, but is not limited to, reagents for size selection of specific size nucleic acids, RT for cDNA synthesis, real-time PCR, dPCR, ddPCR, sanger sequencing, microarray analysis, RNA sequencing, bisulfite sequencing, NGS, etc.

In another example, specific kits might be developed for size selectively separate nucleic acids as required prior to previous applications (such as NGS . . . ). Such kit might comprise mesoporous silica beads, with specifically designed pore sizes, and various reagents for selection of specific size nucleic acids. In such case, the mesoporous layer, the reagents and the procedure are optimized to selectively isolate specific size nucleic acids. Depending on the targets of the size selection (type of nucleic acid, size of interest . . . ), mesoporous silica beads might have been optionally functionalized with carboxyl groups. In such case, a mixture of PEG (with various molecular weight, including PEG8000) and salts (such as NaCl)) might be included in the buffers used. Such size selective kits represent a key method to selectively remove unwanted small DNA/RNA fragments (e.g., adapter dimers, primers), nucleotides, as well as other contaminations and small molecules that could affect the subsequent analysis. The kits may be sold as stand-alone products or as part of an integrated system. In the latter case, the size selection kit may be paired with additional components to facilitate nucleic acid analysis using various biomolecular techniques. This may include, but is not limited to, real-time PCR, dPCR, ddPCR, sanger sequencing, microarray analysis, RNA sequencing, bisulfite sequencing, NGS, etc.

Kits components vary depending on the specific applications of chromatography. For example, a kit for affinity chromatography can include mesoporous silica beads with various additional surface modifications and corresponding buffers. Here, mesoporous silica beads can be coated with various affinity ligands or receptors depending on interested targets. These ligand-receptor pairs can be for example, SAv-biotin, antibodies-antigen, protein A/G-antibodies, enzymes-substrates/inhibitors, etc. Loading and elution buffers can vary depending on the applications, i.e. the type of samples, the type of elution etc. In general, loading buffers are water-based solutions with optional additional ingredients, adjustable pH value and salt concentrations. These can be but not limited to phosphate buffer, Tris buffer etc. Elution buffers can vary strongly depending on the type of elution. For isocratic elution, the loading buffer can be used as elution buffer. For non-specific elution, a buffer with changed pH or salt concentration will be applied. Some common ones are for example, glycerin (highly acidic), Tris-SDS buffer, urea buffer, triethylamine (TEA, highly alkaline), etc. For biospecific elution, competing agent for either stational phase materials or targets can be applied into elution solutions for either normal or reverse elution. For instance, streptavidin molecules captured by biotin-modified mesoporous silica beads can be eluted by adding excess of free biotin molecules into the mixture.

For example, for reverse phase chromatography, a kit can include mesoporous silica beads with modified hydrophobic surface for separating hydrophobic synthetic and biological macromolecules such as drugs, proteins, peptides etc., companied with loading and elution reagents. Loading buffers are commonly hydrophilic solutions with adjustable pH and salt concentration, like water-based buffers such as phosphate buffer, tris buffer, etc. Elution buffers can be organic solvents such as methanol, ethanol, n-propanol, acetonitrile etc., which can also contain chaotropic agents such as GITC, surfactants such as SDS and Tween, ion-pairing agent such as trifluoroacetic acid (TFA), acetic acid (Hac), formic acid (FA), etc., salt such as ammonium acetate or ammonium formate etc. pH values can be adjusted if needed.

### EXAMPLES

The following Examples are illustrative and do not limit the disclosure.

### Example 1: Rigid and Chemical Resistant Materials for Producing Shear Device and Pre-Mixer

In this example, rigid and chemical resistant materials stainless steel 316L or PEEK (polyetheretherketone) polymer, or fluoropolymer is used to build shear device and pre-mixer.

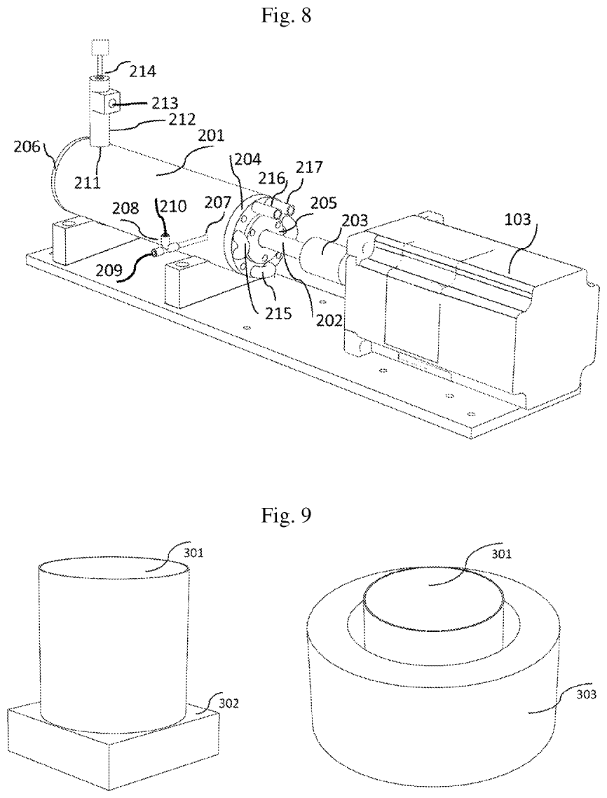

As illustrated in FIG. 6 and FIG. 8, a complete shear device is consisted of a concentric stator 201 and rotor 202 assembled by shaft bearings and seals accommodated inside housing 205. The rotation of rotor is controlled by a servo motor through shaft 202. The gap between stator and rotor is from 1 micron to 200 microns. The surface roughness of stator and rotor is 0.4 microammeter Ra. An inlet port 207 and an outlet port 211 with 1 mm diameter through hole on two ends of the stator. In another example, two inlet ports arranged orthogonally on the stator are used. A thermometer housing 212 is connected directly with outlet of shear device. A thermometer probe 214 is installed inside the housing. The housing has at least one outlet 213 to allow emulsion flow out.

In an example as shown in FIG. 7, eight TCF channels 218 are constructed through the wall of stator of shear device and connected in a close loop through TCF manifold 215 to temperature controller (chiller or heater). Water or oil is used as TCF and circulated inside TCF channels by a pump.

In an example, PEEK polymer, or fluoropolymer, or stainless steel made pre-mixers are used to create emulsions before introducing into shear device for process. As illustrated in FIG. 6, T-shape pre-mixer 208a, Y-shape pre-mixer 208b, and cross-shape pre-mixer 208c with through hole 0.1 mm to 0.5 mm are used.

In an example, the magnetic beads solution from shear device (after evaporation of organic solvent), are dispensed in 1-litter centrifuge bottles. The beads solution is stirred and sonicated for 5 minutes. A swinging out centrifuge is used to centrifuge the beads solution at centrifugation rotation speed of 1000 RPM for 10 minutes. After centrifugation, the supernatant is removed by pouring out. The remaining beads were re-dispensed in 0.25% SDS aqueous solution. The centrifugation is repeated for three times. Once completed, the selected magnetic beads have uniform diameter of 800 nm with coefficient of variation less than 10%.

In an example, the magnetic beads in 0.25% SDS aqueous solution is collected in a 2-litter glass beaker, stirred and sonicated. Then the beaker is placed on a top of permanent magnet (120 mm in diameter 30 mm in height) and the magnetic beads are captured on the bottom of the beaker for 2 minutes. After capturing, the supernatant solution is poured out of the beaker while the beaker is still on the magnet. In this example, after magnetic wash, the magnetic beads that are smaller than 800 nm in diameters are removed out of initial beads solution.

In another example, a ring magnet for 1-Litter beaker is used for magnetic wash. The separation duration is 1 minute, and the selected magnetic beads are captured on the side wall of the beaker after separation and the supernatant solution is removed out of the beaker by using a suction apparatus.

The magnetic wash can be carried out for multiple times to get more uniform magnetic beads.

### Example 2: Oleic Acid Coated Iron Oxide Nanoparticles

In this example, oleic acid coated iron oxide nanoparticles are produced by using co-precipitation method.

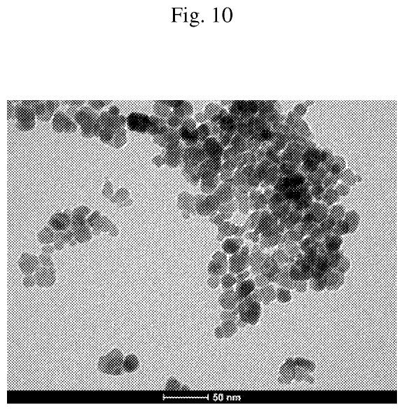

The method uses mixed solution of iron chloride hexahydrate and iron chloride tetrahydrate (molar ratio 1.5:1) and react with ammonia hydroxide under oxygen-free and heated environment to produce superparamagnetic iron oxide nanoparticles. The oleic acid is used to coat the nanoparticle to make hydrophobic nanoparticle during the reaction. After several wash steps by using water and solvent (such as ethanol or acetone), the nanoparticles are suspended in organic solvent (with certain nanoparticle concentration) that has high volatility and immiscible with aqueous solution, such as cyclohexane, pentane, or toluene. In one example, the produced iron oxide nanoparticles are around 15 nm in diameter as shown in FIG. 10.

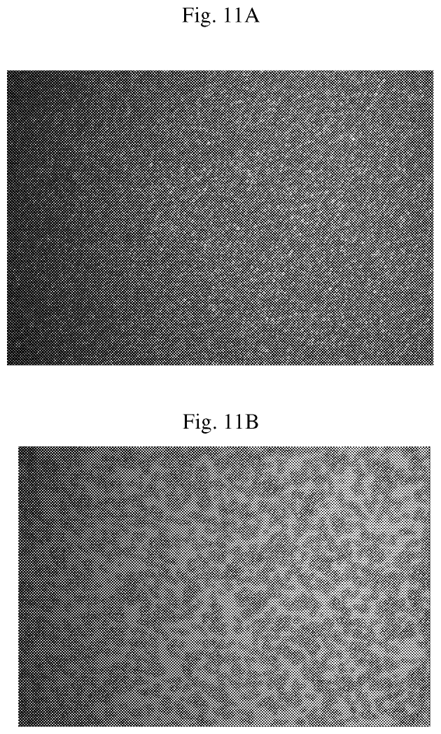

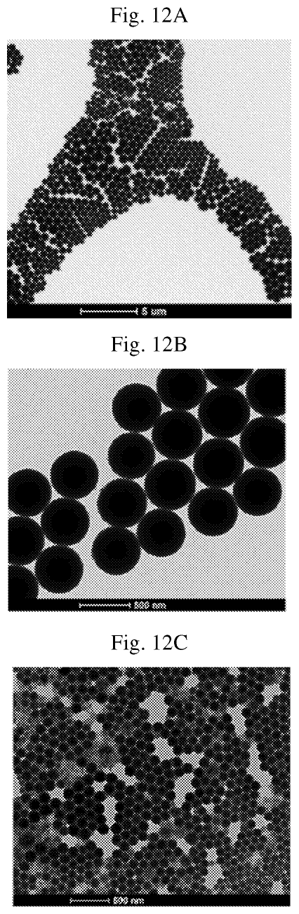

In one example, 1-10% PVP and 1-4% SDS aqueous solution are used to form aqueous solution for emulsification. In one example, 4% PVP and 4% SDS aqueous solution is used to produce the magnetic beads ranged from 400 nm to 1000 nm in diameter. The CV of diameter of the beads is less than 15%. In this example, the emulsion temperature is maintained at 22° C. The produced emulsion droplets from the shear device are highly uniform (as shown in FIGS. 11A-11B) with high production yield (more than 6:10 of ratio between total mass of produced beads vs the total mass of nanoparticles used). The produced emulsion droplets subsequently result uniform magnetic beads, as shown in FIGS. 12A-C.

In another example, 5% PVP and 4% SDS aqueous solution is used to produce the magnetic beads ranged from 50 nm to 400 nm in diameter. The emulsion temperature is controlled to be from 5 to 10° C. FIG. 12C shows the produced uniform magnetic beads 200 nm in diameters.

### Example 3: Test Parameters for Emulsification Using Shear Device

In another examples, certain combinations of parameters used for emulsification using shear device are presented. The dimension of magnetic emulsion (droplet) and magnetic bead (the magnetic bead is created after evaporation of droplet) is related to the parameters of emulsification and shear device process. Table 1 below shows resulting diameters of droplets and beads for different combinations of parameters used for emulsification.

TABLE 1
Dimensions of droplet and beads using different emulsification parameters. Gap between rotor and
stator of shear device - 0.01-0.02 microns. Sodium dodecyl sulfate (SDS), Polyvinylpyrrolidone
(PVP) 1.3 million molecular weight. Ferrofluid: magnetic nanoparticles in organic solvent.

| | | | Flow | | | | |
|---|---|---|---|---|---|---|---|
| | | | rate | | | | Average |
| | | | ratio of | | | | bead |
| | Concentration | Composition | ferrofluid | Flow | | Average | diameter- |
| Rotation | of | of | to | rate of | | droplet | after |
| speed | ferrofluid | Aqueous | aqueous | ferrofluid | Temperature | diameter | evaporation |
| (RMP) | (mg/mL) | fluid | fluid | (mL/min) | (° C.) | (micrometer) | (micrometer) |
| 100 | 10-30 | 1-4% | 0.5-1.5 | 0.5-2 | 22 | 6 | 1.3 |
| | | SDS and | | | | | |
| | | 2- | | | | | |
| | | 4.5% PVP | | | | | |
| 200 | 10-30 | 1-4% | 0.5-1.5 | 0.5-2 | 22 | 5 | 0.9 |
| | | SDS and | | | | | |
| | | 2- | | | | | |
| | | 4.5% PVP | | | | | |
| 300 | 10-30 | 1-4% | 0.5-1.5 | 0.5-2 | 22 | 4 | 0.8 |
| | | SDS and | | | | | |
| | | 2- | | | | | |
| | | 4.5% PVP | | | | | |
| 400 | 10-30 | 1-4% | 0.5-1.5 | 0.5-2 | 22 | 3 | 0.4 |
| | | SDS and | | | | | |
| | | 2- | | | | | |
| | | 4.5% PVP | | | | | |
| 500 | 10-30 | 1-4% | 0.5-1.5 | 0.5-2 | 22 | 2.5 | 0.3 |
| | | SDS and | | | | | |
| | | 2- | | | | | |
| | | 4.5% PVP | | | | | |
| 600 | 10-30 | 1-4% | 0.5-1.5 | 0.5-2 | 22 | 2.5 | 0.3 |
| | | SDS and | | | | | |
| | | 2- | | | | | |
| | | 4.5% PVP | | | | | |
| 700 | 10-30 | 1-4% | 0.5-1.5 | 0.5-2 | 22 | 2.5 | 0.3 |
| | | SDS and | | | | | |
| | | 2- | | | | | |
| | | 4.5% PVP | | | | | |
| 800 | 10-30 | 1-4% | 0.5-1.5 | 0.5-2 | 22 | 2.2 | 0.2 |
| | | SDS and | | | | | |
| | | 2- | | | | | |
| | | 4.5% PVP | | | | | |
| 900 | 10-30 | 1-4% | 0.5-1.5 | 0.5-2 | 22 | 1.8 | 0.15 |
| | | SDS and | | | | | |
| | | 2- | | | | | |
| | | 4.5% PVP | | | | | |
| 1000 | 10-30 | 1-4% | 0.5-1.5 | 0.5-2 | 22 | 1.8 | 0.15 |
| | | SDS and | | | | | |
| | | 2- | | | | | |
| | | 4.5% PVP | | | | | |
| 400 | 5-20 | 1-4% | 0.5-1.5 | 0.5-2 | 22 | 2.5 | 0.3 |
| | | SDS and | | | | | |
| | | 5- | | | | | |
| | | 10% PVP | | | | | |
| 500 | 5-20 | 1-4% | 0.5-1.5 | 0.5-2 | 22 | 2.3 | 0.25 |
| | | SDS and | | | | | |
| | | 5- | | | | | |
| | | 10% PVP | | | | | |
| 600 | 5-20 | 1-4% | 0.5-1.5 | 0.5-2 | 22 | 2.2 | 0.2 |
| | | SDS and | | | | | |
| | | 5- | | | | | |
| | | 10% PVP | | | | | |
| 700 | 5-20 | 1-4% | 0.5-1.5 | 0.5-2 | 22 | 2 | 0.16 |
| | | SDS and | | | | | |
| | | 5- | | | | | |
| | | 10% PVP | | | | | |
| 800 | 5-20 | 1-4% | 0.5-1.5 | 0.5-2 | 22 | 1.5 | 0.12 |
| | | SDS and | | | | | |
| | | 5- | | | | | |
| | | 10% PVP | | | | | |
| 900 | 5-20 | 1-4% | 0.5-1.5 | 0.5-2 | 22 | 1.2 | 0.1 |
| | | SDS and | | | | | |
| | | 5- | | | | | |
| | | 10% PVP | | | | | |
| 1000 | 5-20 | 1-4% | 0.5-1.5 | 0.5-2 | 22 | 1.2 | 0.1 |
| | | SDS and | | | | | |
| | | 5- | | | | | |
| | | 10% PVP | | | | | |
| 300 | 10-30 | 1-4% | 0.5-1.5 | 0.5-2 | 10 | 3 | 0.4 |
| | | SDS and | | | | | |
| | | 2- | | | | | |
| | | 4.5% PVP | | | | | |
| 300 | 10-30 | 1-4% | 0.5-1.5 | 0.5-2 | 15 | 3 | 0.4 |
| | | SDS and | | | | | |
| | | 2- | | | | | |
| | | 4.5% PVP | | | | | |
| 300 | 10-30 | 1-4% | 0.5-1.5 | 0.5-2 | 20 | 4 | 0.8 |
| | | SDS and | | | | | |
| | | 2- | | | | | |
| | | 4.5% PVP | | | | | |
| 300 | 10-30 | 1-4% | 0.5-1.5 | 0.5-2 | 30 | 5 | 0.9 |
| | | SDS and | | | | | |
| | | 2- | | | | | |
| | | 4.5% PVP | | | | | |
| 300 | 10-30 | 1-4% | 0.5-1.5 | 0.5-2 | 40 | 6 | 1.3 |
| | | SDS and | | | | | |
| | | 2- | | | | | |
| | | 4.5% PVP | | | | | |
| 400 | 5-20 | 1-4% | 0.5-1.5 | 0.5-2 | 5 | 1.5 | 0.1 |
| | | SDS and | | | | | |
| | | 5- | | | | | |
| | | 10% PVP | | | | | |
| 400 | 5-20 | 1-4% | 0.5-1.5 | 0.5-2 | 10 | 2.2 | 0.2 |
| | | SDS and | | | | | |
| | | 5- | | | | | |
| | | 10% PVP | | | | | |
| 400 | 5-20 | 1-4% | 0.5-1.5 | 0.5-2 | 15 | 2.5 | 0.3 |
| | | SDS and | | | | | |
| | | 5- | | | | | |
| | | 10% PVP | | | | | |

In another example, PVP, PVA, Gelatin, PEG, and SDS were used for emulsification to generate magnetic droplets and beads. The composition of aqueous fluid vs. resulted dimensions of droplets and beads are listed in Table 2 below.

TABLE 2
Composition of aqueous fluid vs. diameters of droplets and beads.

| Composition of | | Average bead |
|---|---|---|
| Aqueous fluid (weight | | diameter after |
| percentage in purified | Average droplet | evaporation |
| water) | diameter (micrometer) | (micrometer) |
| 4% PVP, 2% SDS | 4 | 0.8 |
| 2% PVA, 2% SDS | 4 | 0.8 |
| 4% Gelatin, 2% SDS | 3 | 0.4 |
| 10% PEG, 2% SDS | 3 | 0.4 |
| 30% PEG, 2% SDS | 5 | 0.9 |

Chemicals used in the examples: Ferrofluid (nanoparticles in cyclohexane), Polyvinylpyrrolidone (PVP, molecular weight 1.3 million), dextran, Polyvinyl alcohol (PVA, 88% hydrolyzed, average molecular weight 145,000 to 180,000); Polyethylene glycol (PEG, average molecular weight 8000), Gelatin, Sodium dodecyl sulfate (SDS), water.
Shear device parameters used to form emulsions (droplets): Concentration of ferrofluid (10 mg/mL), Rotation 300 RPM, Flow rate of ferrofluid: 1 mL/min, Flow rate of aqueous fluid: 1 mL/min, temperature: 22° C.

In another example, the centrifugation parameters are listed for producing magnetic beads with different diameters, as shown in Table 3 below.

TABLE 3
Parameters used for producing magnetic
beads with different diameters.

| Nominal | | | | |
|---|---|---|---|---|
| diameter of | | | | |
| Beads | | | | |
| suspension | | | | Average bead |
| used for | Rotation | Relative | | diameter after |
| centrifugation | speed | centrifugal | Centrifugation | centrifugation |
| (micrometer) | (RPM) | force (g) | time (minute) | (micrometer) |
| 0.2 | 3500 | 726 | 15 | 0.2 |
| 0.4 | 2800 | 465 | 15 | 0.4 |
| 0.8 | 1400 | 116 | 15 | 0.8 |

Centrifugation for 1 L beads suspension by swinging out centrifuge. After each centrifugation process, the supernatant is removed and centrifugation pellets (on the bottom of centrifugation bottle) is retained and measured.

In another example, the parameters for producing magnetic beads with different diameters by using magnetic separation are listed in Table 4 below.

TABLE 4
Parameters used for producing magnetic
beads by using magnetic separation.

| Nominal diameter of Beads | Separation | Average bead |
|---|---|---|
| suspension used for magnetic | time | diameter |
| separation (micrometer) | (minute) | (micrometer) |
| 0.2 | 10 | 0.2 |
| 0.4 | 5 | 0.4 |
| 0.8 | 2 | 0.8 |

A permanent magnet is used for separation. The beads suspension in a 1 L beaker is from 0.5 L to 1 L. After magnetic separation process, the supernatant is removed and discarded. The remaining beads in the beaker are measured.

### Example 4: Silica Layers Coating

Following examples of silica layers coating are all based on spherical 800 nm superparamagnetic beads made by Magnostics. In an example for coating silicate around magnetic beads, magnetic beads were washed with 0.0005 w/v % SDS aqueous solution to improve the absorption of silicate. Sodium silicate with the weight ratio of silicate to beads of 40:1 was dissolved in purified water with a concentration of 16.67% w/v. Sodium silicate is ion-exchanged via ion-exchange resin and titrated with ammonium hydroxide to approximately pH 9.5. Initially, while using periodical ultrasonic homogenizer for the bead's reaction vessel, half amount of the treated silicate solution was added to the vessel with vigorous stirring at room temperature for 90 minutes. After the reaction, the other half of the silicate solution was added to the reaction vessel and the whole was reacted for another 90 minutes.

In an example for coating first silane layer over silicate, which was coated as described above, the beads were washed with purified water and subsequently with ethanol using magnetic wash procedure mentioned above. They were then suspended in a 70% (v/v) ethanol aqueous solution titrated with ammonium hydroxide to about pH 9. To improve the homogeneity of TEOS on the surface while preventing the immediate hydrolysis prior to the binding, TEOS with the volume to weight (v/w) ratio to beads of 0.08% was dissolved in ethanol and then added dropwise (for example, at 21.5 L/min for pure TEOS effectively) to the beads solution. The beads mixture was stirred vigorously at room temperature for approximately 20 hours.

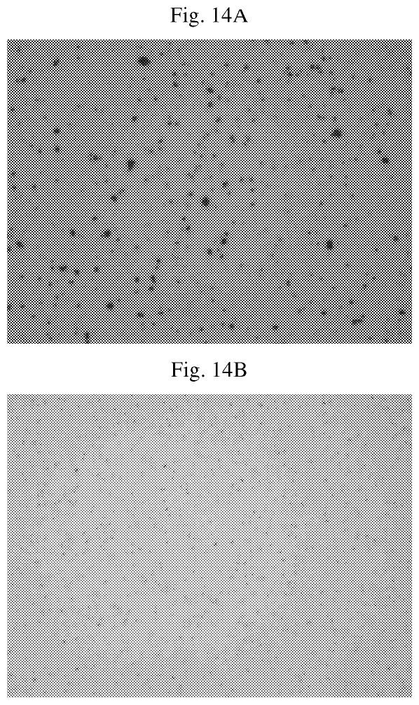

When the beads were washed by using strong magnet during the process, the beads aggregated as shown in FIG. 14A. Therefore, an alternative wash process was used to prevent the formation of aggregates and produce monodispersed beads as shown in FIG. 14B. To do that, the beads were washed with alkalic purified water using magnetic wash procedure with very weak magnetic separation strength. In addition, the mixture was stirred vigorously at room temperature with additional periodical ultrasonication using an ultrasonic homogenizer and reacted for 20 hours.

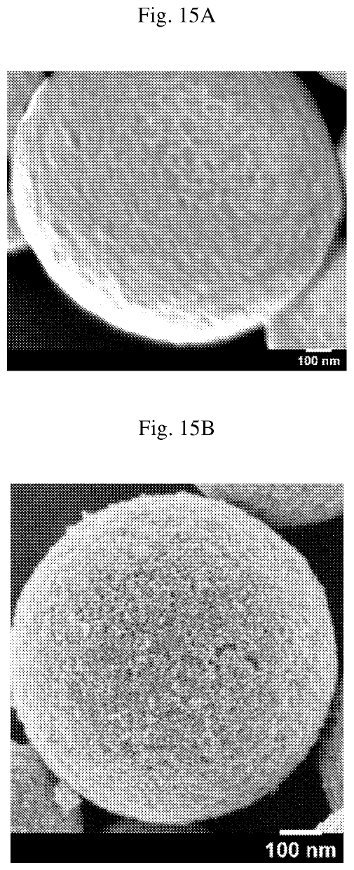

The roughness of the coated silica layer can be adjusted by adding TEOS at different rate of velocity to concentration of beads into the mixture. In an example, TEOS was effectively added at rate of 21.5 μL/min. Here, a minute molar ratio between TEOS to beads in the vessel was 1.2·10⁷:1. After reaction, a smooth silica layer was coated on the beads as shown in FIG. 15A. In another example, TEOS was effectively added at rate of 5 μL/min corresponding to a minute molar ratio between TEOS and beads of 2.7·10⁶:1, the surface roughness of the outer silica layer was significant higher, as shown FIG. 15B.

### Example 5: Coating a High Surface-to-Volume Ratio Silane Layer

In an example for coating a high surface-to-volume ratio silane layer, the beads with polymerized TEOS (coated as mentioned above) were washed with purified water, and activated with acid such as but not limited to 2 Molar nitric acid or 1 Molar hydrochloride acid, followed by another two washes with purified water. CTAB and urea were dissolved in purified water and thereafter mixed with the beads. Cyclohexane and 1-pentanol were added to the mix. The molar ratio was CTAB:urea:water:cyclohexane is 0.27:1:166.6:27.7 and the concentration of 1-pentanol was 1% v/v. The mix was stirred for 30 minutes, and then TEOS pure was added dropwise (for example, at a rate of 20 μL/min) into the mixture. The reaction duration was approximately 20 hours. Thereafter, the beads were washed with ethanol and stirred in 1 molar hydrochloric acid (in ethanol) for 30 minutes to remove the CTAB templates. After the removal of CTAB, the beads were washed and suspended in ethanol or purified water.

In another example, ultrasonication under stirring at room temperature was added during the reaction. From this change in procedure, additional high surface-to-volume ratio surface structure, with corrugated surface or mesoporous morphology, of silica layers were not formed on the magnetic beads. The thickness of the silica layer was about 10-20 nm, corresponding to the silane layer mentioned in Example 4.

In another example, the reaction mentioned above was carried out at 70° C. without using ultrasonication. The concentration of beads was 0.1 mg/mL, TEOS was added at a ratio of 2.5 μL per mg of beads and urea was used as catalyst. A non-homogenous silica surface on the beads was formed with many silica bumps. we hypothesize a that TEOS condensed prior to binding to magnetic beads, as a result, TEOS formed silica bumps on the beads. This might indicate that, in this example, the concentration of beads in the reaction was too low.

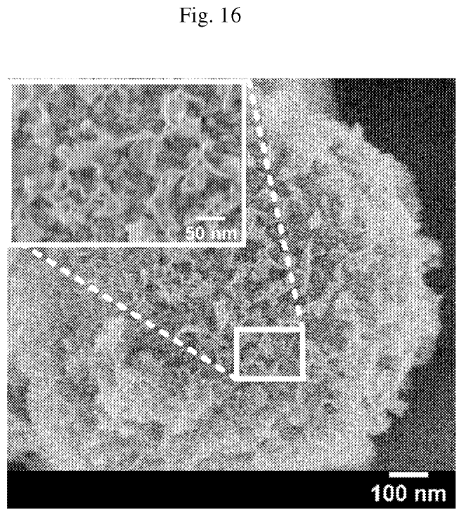

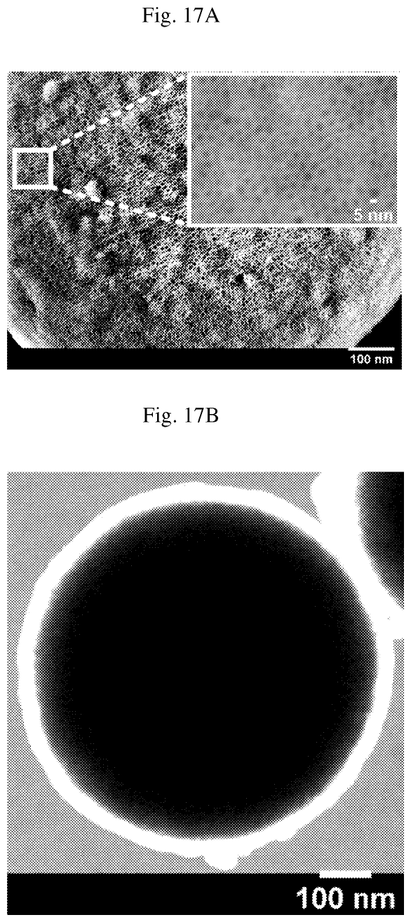

In another example, previously mentioned process was carried out, but the concentration of beads was increased to 8 mg/mL instead, TEOS was added at a ratio of 2 μL per mg of beads and urea was used as catalyst. SEM image in FIG. 16 shows corrugated silica sheets loosely coated. The silica layers obtained from these types of coating are named as Loose Silica (LS) in this application.

In another example, the reaction mentioned above was carried out, but the concentration of beads was adjusted to 5 mg/mL, TEOS was added at a ratio of 10 μL per mg of beads, while ammonium hydroxide was used as catalyst instead of urea. Thicker wrinkles of silica sheets were observed on the beads produced. With an appropriate/optimized ratio of all involved chemical components, a homogenous and dense mesoporous morphology can be achieved, with a dense contribution of the bi-continuous microemulsion.

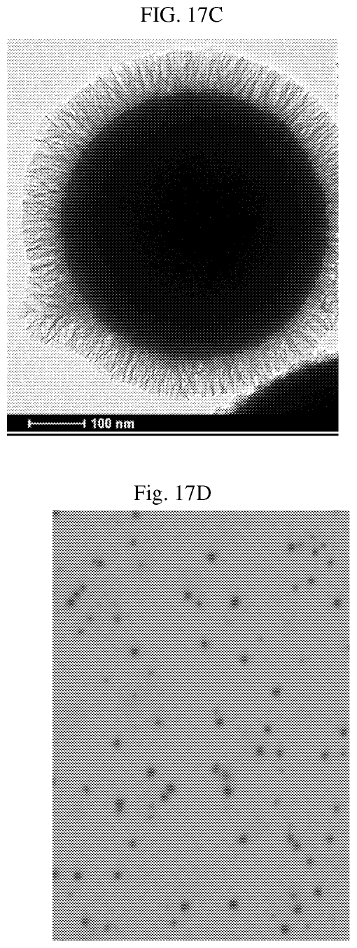

In another example, 5 μL TEOS per mg beads was used in coating process, with periodic sonication at a power of 24 watt. After coating, the beads were fully coated with homogenous mesoporous silica with thin wrinkles (white colour) and small round pores (dark colour) as shown in FIG. 17A. The thickness of the total silica layers on the beads might be estimated about 50 nm as shown in FIGS. 17A-17B (outer white colour), including 20% of the first silane layer. In addition, the beads were monodispersed without aggregation. In another example, the coating process was carried out similarly, but without using sonication. A similar homogenous mesoporous morphology was formed on those beads, however, those beads were aggregated. The silica layers obtained from these processes, have round pores with about 5 nm in diameters and are named as Small Pores (SP) in this application, as shown in FIG. 17A insert. In another example, sonication power of 60 watt was used in the coating process. After completion, the mesoporous silica sheets on the beads were damaged by the strong sonication power used (e.g., certain portion of silica sheets were removed from the beads surface). In conclusion, a proper sonication power can be used in the coating process to prevent the beads from aggregating (FIG. 17D), whilst ensure the creation of a dense and homogenous mesoporous morphology of silica on the beads surface. FIG. 17C illustrates a TEM image of SP using accelerating voltage of 200 kV. The thickness of the total silica layer is shown as about 50 nm with the pore depth of around 40 nm.

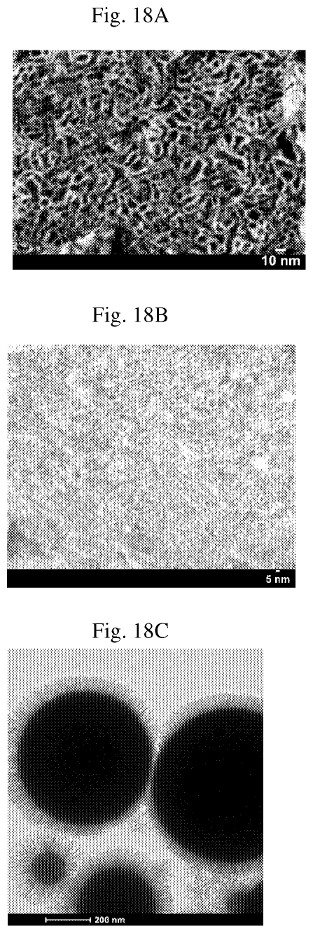

In another example, 5% v/v of 1-pentanol was used. After completion, the silica layers with larger (about 10 nm in widths) oval pores were formed on the beads, as shown in FIG. 18A. The silica layers obtained from these processes are named as Large Pores (LP) in this application. In another examples, 2-propanol was used as replacement of 1-pentanol. In this case, a more dense mesoporous structure of silica layer was formed on the beads, as shown in FIG. 18B. The thickness of the overall silica sheets on the beads remained similar compared to the ones coated using 1-pentanol, but with a different dendritic fibrous-like morphology of the pores.

In another example, twice the amount of CTAB was added into the reaction mixture. Moreover, TEOS was premixed with small amount of cyclohexane (5 mL) and added into the mixture in two times, with a time distance of 90 min. A mesoporous silica layer were observed as shown in FIG. 18C.

For example, the specific surface area (ratio of surface area to mass of beads) of three types of silica coated magnetic beads were measured by Brunauer-Emmett-Teller (BET) surface area analysis. As shown in Table 5, the beads coated with large pore (LP) and small pore (SP) silica layers of 40 nm nominal thickness have higher specific surface areas than that of the beads coated with only the smooth silica layer (SM). In this case, it appears that mesoporous LP beads larger surface area than mesoporous SP beads. These data demonstrate that both the LP and SP mesoporous beads have 40-50 times higher surface areas than SM beads, providing higher binding capacity. The ability to customize the pore structure on the silica surface impacts the apparent surface area. The selection of the mesoporous silica coating makes it possible in principle to select specific nucleic acids, proteins, and molecules.

TABLE 5
Specific surface area of different silica coated magnetic beads
(LP: large pores mesoporous silica beads, SP: small pores
mesoporous silica beads, SM: smooth silica beads) measured
by Brunauer-Emmett-Teller (BET) surface area analysis. The
thickness of the mesoporous layers was approximately 40 nm.

| | LP | SM | SP |
|---|---|---|---|
| BET Surface Area (m²/g) | 168.13 | 3.76 | 134.06 |

### Example 6: Mesoporous Silica Beads, with High Surface-to-Volume Ratio, can be Further Modified with Amine, Carboxylic Acid or Streptavidin (SAv)

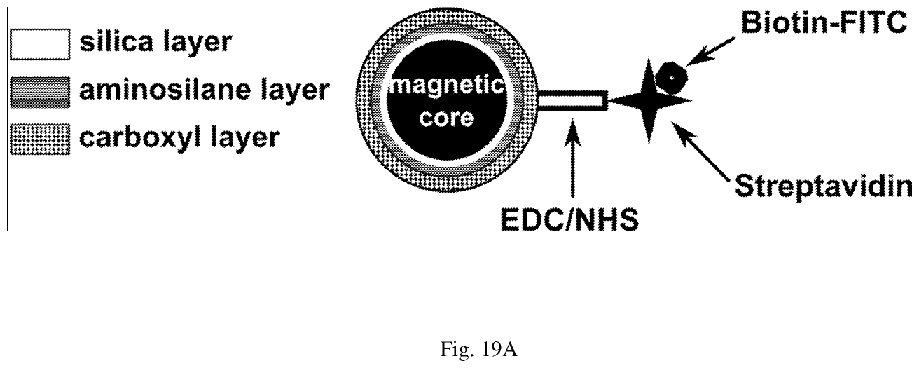

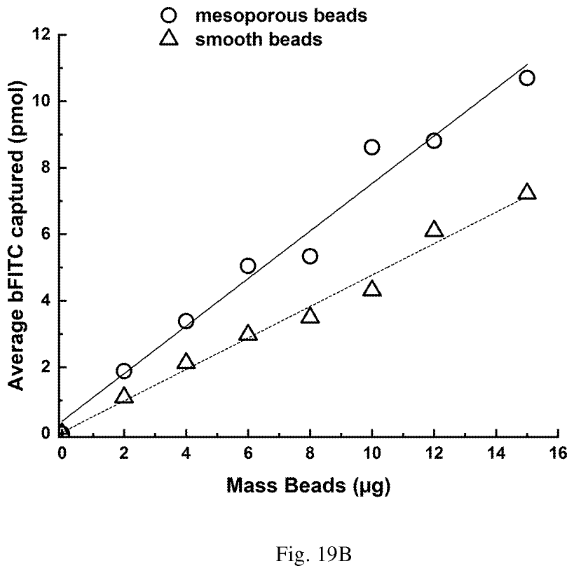

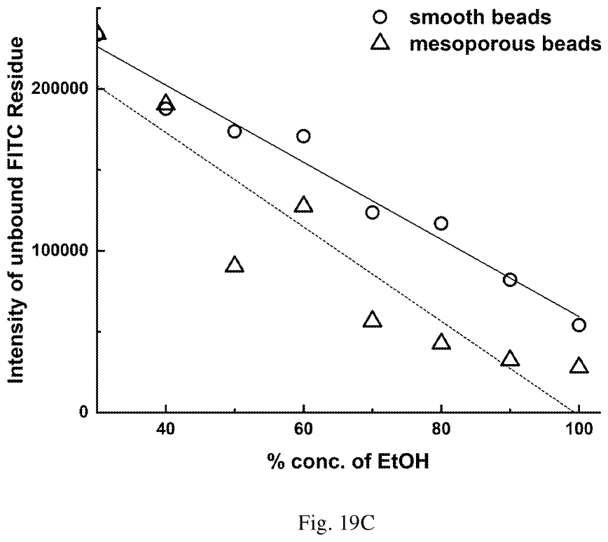

In an example, SM beads and LS beads (produced as per Example 5), with high surface-to-volume ratio, were used for the further modification with amine, carboxylic acid or streptavidin (SAv). The amine groups from the aminosilane (3-Aminopropyl)triethoxysilane (APTES) were covalently bound to the silanol groups of the silica surface. In addition, carboxyl groups were further bound with amine groups. The mesoporous carboxyl beads produced were then firstly activated using 1-Ethyl-3-(3-dimethylaminopropyl) carbodiimide (EDC)/sulfo-N-hydroxysuccinimide (sNHS) chemistry, before covalent coating of the mesoporous beads with SAv. To evaluate the improvement in binding capacity of the LS beads compared to normal SM beads, and due to an affinitive specific interaction between biotin and SAv on the mesoporous beads, as shown in FIG. 19A, the SAv-modified SM and LS beads were incubated with biotin-fluorescein. After 30 min incubation at room temperature (to allow for maximum biotin-fluorescein molecules to be captured by the SAv beads according to available surface), the magnetic beads were removed from the solution and the fluorescence intensity of the supernatant (corresponding to unbound/residue of biotin-fluorescein) was measured and the biotin binding capacity was calculated (FIG. 19B). These data highlight that the biotin binding capacity of the SAv beads was improved by over 100% for LS beads compared SM beads. This confirms that the corrugated silica layer of LS beads creates a considerably large specific surface area which can highly improve the binding/absorption of biochemical molecules on the beads.

In another example, SAv was coated on SP silica beads, as per description above. The biotin binding capacity didn't increase significantly compared with SAv-modified SM beads and was lower than for both SAv-modified LP beads and SAv-modified LS beads. These results revealed that quantity of streptavidin conjugated to the SP beads is the same as of SM beads, despite the higher surface area. This result can be explained by the pore size of SP beads, which is too small for SAv molecules to enter. This is an example of size selective binding of the mesoporous materials.

In another example, 20 μg of mesoporous SP and smooth SM silica coated magnetic beads were incubated with 1 μL of 25 μM fluorescein isothiocyanate (FITC, size around 2 nm×2 nm) in water-ethanol mixtures of different ethanol concentration (v/v). After incubation (to allow for maximum FITC molecules to be captured by the beads according to available surface), the magnetic beads were removed from the solution and the fluorescence intensity of the supernatant (corresponding to unbound/residue of FITC molecule) was measured, as shown in FIG. 19C. The fluorescence intensity of unbound FITC is inversely proportional to the quantity of FITC molecules captured by the beads. We can conclude that more FITC molecules are captured by the mesoporous SP beads than by the smooth SM beads, which is a example of size selective chromatography.

These results highlight that the pore size on the beads can be used to target molecules of specific size using affinity and silica surface chromatography.

### Example 7: Extraction of Nucleic Acids Using Silica Coated Magnetic Beads in Specific Buffers

In this example, the extraction (or capturing) capacity of different types of silica layer magnetic beads for different nucleic acid-related molecules (such as nucleotides, nucleotide oligomers and long chain nucleic acids (Poly(A) and λ-DNA)) were demonstrated and compared. In more details, dATP (5 Å×7 Å in dimensions), (dT)₁₈ oligomers (single-stranded 18-mer oligonucleotide with repeated thymine bases, 0.5 nm×12 nm in dimensions as linear chain), Poly(A) (RNA, 2,100-10,000 nt) or λ-DNA (dsDNA, 48,502 bp) were captured by silica coated beads. To do that, a specific amount of nucleic acids were incubated with SM beads, mesoporous LP beads or mesoporous SP beads in 70% (v/v) ethanol in the presence of different salt concentration at 4° C. After incubation, the nucleic acid-beads mixture was washed with 70% cold ethanol. The nucleic acids were then eluted in different elution buffer and the magnetic beads were removed. The concentration of recovered nucleic acids were measured by UV-visible spectrophotometry (UV-Vis) and recovery efficiency of each type of beads, for each type of nucleic acids, was calculated by comparing original quantity and eluted quantity.

In one example, dATP was incubated and captured by SM silica beads at different MgCl₂ concentrations. It shows that higher salt concentration increases the capture efficiency of silica coated magnetic beads for nucleotides.

In another example, long chain nucleic acids Poly (A) was incubated and captured by either SM silica beads or mesoporous LP silica beads. SM silica beads have much higher recovery efficiency for Poly (A) than LP beads, for all range of beads quantity. It suggests that, although the specific surface area of mesoporous silica beads is very high, the dimensions of the pores might restrict the absorption of certain large nucleic acids. In some applications, this unique feature can be used to selectively screen or capture nucleic acids (or other molecules) according to their dimensions.

In another example, 18-mer oligo primers were incubated and captured by either mesoporous LP silica beads or mesoporous SP silica beads, at different MgCl₂ concentrations. The recovery efficiency increases dramatically for both types of beads as divalent salt concentration increased, until the recovery efficiency reaches a maximum and saturates at MgCl₂ concentrations of about 200 mM. In addition, mesoporous LP silica beads present slightly better recovery efficiency for the 18-mer oligo primers than SP beads.

In another example, three nucleic acid-related molecules of different dimensions (dATP, 18-mer dT oligo-primers and 2-DNA) were incubated and captured by mesoporous LP silica, mesoporous SP silica, and smooth SM silica beads in the presence of 1M MgCl₂. The recovery rate for each type of molecules was different according to the type of beads, with a demonstrated selective behaviour according to the silica surface structure. For large nucleic acids, mesoporous LP beads have a low recovery efficiency for 2-DNA. For small nucleic acid-related molecules, mesoporous LP silica beads demonstrate the highest recovery efficiency (i.e., the highest capture capacity) for both nucleotides and 18-mer oligo primers. Mesoporous SP silica beads have higher recovery efficiency for nucleotides and 18-mer oligo primers than smooth SM silica beads. These results are consistent with the specific surface area of these three types of silica coated beads as shown by BET measurement (Table 5). Therefore, we believe that for small molecules that can get inside the pores, a higher specific surface area for the silica beads allows for higher absorption and capturing. Taken together, the results from this example confirm the selective capturing ability of mesoporous silica beads for this important class of biomolecules based on their size.

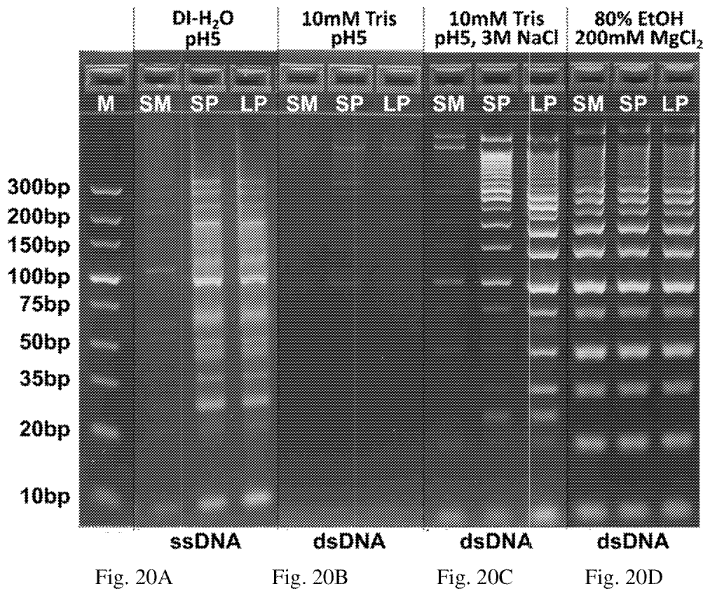

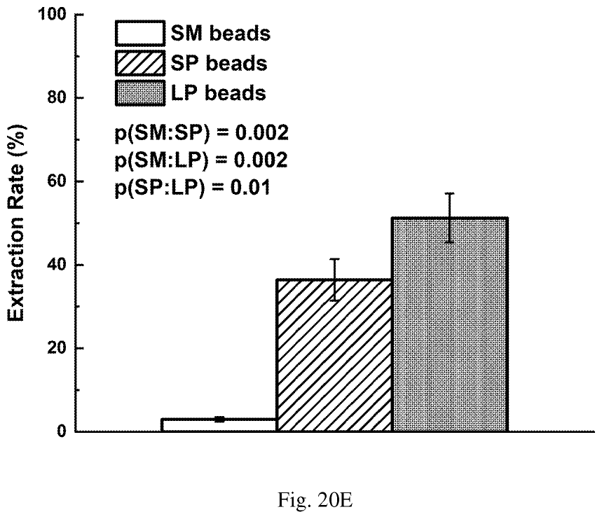

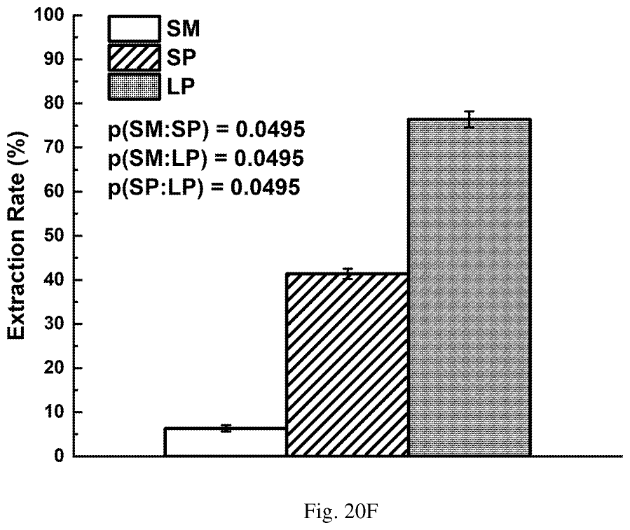

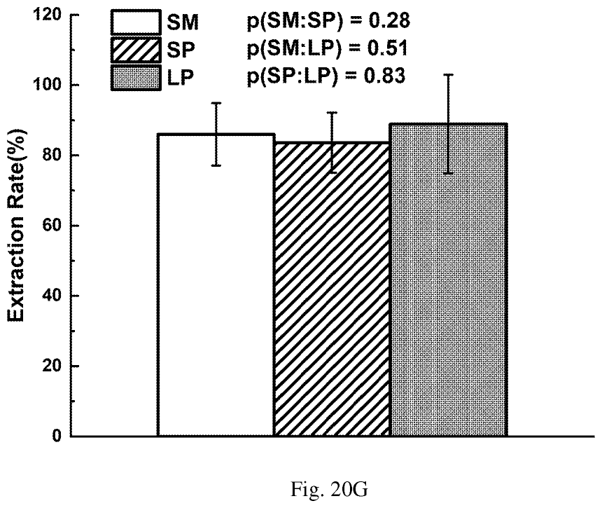

In one example, dsDNA ladders with different lengths of DNA fragments (ranging from 10 to 2,500 base pairs (bp)) were incubated with smooth SM silica, mesoporous SP silica or mesoporous LP silica beads in DI-water at pH 5. As the dsDNA denatures in DI-water at room temperature, the DNA extracted in this example should have a ssDNA conformation. The results show the migration of eluted DNA on a 4% agarose gel electrophoresis (FIG. 20A) and the percentage of recovery (i.e., the extraction rate) of eluted DNA as measured by UV-Vis measurement (FIG. 20E). The gel shows that mesoporous SP and LP silica coated beads recovered considerably more ssDNA than smooth SM silica beads.

In another example, a dsDNA ladder was spiked into Tris buffer, pH 5. The extraction efficiency was low for all three types of beads (FIG. 20B), with concentrations too low to be measured by UV-Vis. Yet both SP and LP mesoporous beads performed slightly better than SM smooth beads. When additional salt was added to the Tris buffer, i.e., addition of 3M NaCl, an increase in the DNA recovery was observed for the three types of beads. Mesoporous LP beads showed better extraction rate than the other types of beads, especially for short DNA below 100 bp (FIGS. 20C, 20F) (i.e., c, f)).

In another example, the dsDNA ladder was spiked in 80% ethanol solution with 200 mM MgCl₂. In this condition, good recovery efficiency of approximately 80% DNA was achieved, over the range of DNA fragments, as shown in FIGS. 20D, 20G (i.e., d, g). The extraction of short fragments of DNA on silica coated magnetic beads has been achieved with divalent salts (e.g., MgCl₂, CaCl2). A concentration of divalent salt in the range of 150 mM to 6M improves the capture of short fragments up to 150 bp and molecules which are not highly hydrophilic.

The results of statistical Kruskal-Wallis ANOVA test (p-values with 0.05 as the significance level) were shown in FIGS. 20E, 20F, 20G (i.e., e, f, g) for the data of FIGS. 20A, 20C, 20D (i.e., a, c, d). The low p values of the data of FIG. 20A, 20C (a) and (c) indicate a significant improvement of the DNA-recovery by mesoporous beads than that by smooth beads.

This example demonstrates that the mesoporous beads can be used to adsorb both single stranded and double stranded DNA with enhanced efficiencies (relative to smooth silica beads). The enhanced extraction efficiency is associated with the increase surface area of the mesoporous beads but also from chemical bonds. These chemical interactions may be attributed to the nanometre scale features of the pores that enable multivalent binding and/or modify short and long-range bonding. Notably, both mesoporous SP and LP silica coated beads appear to efficiently adsorb single stranded DNAs in water. The double stranded molecules also appear to more efficiently bind to the mesoporous materials in the 3M NaCl aqueous solution in Tris buffer at pH 5.

The fact that small fragments of single and double stranded DNA can be extracted from aqueous solutions is of particular importance for next generation sequencing library preparation applications. The tuneable pore size of mesoporous silica beads can be potentially designed to select specific molecules. In addition, size selective nucleic acid extraction can be achieved via, for instance, varying the volume ratio between alcohol and the rest. This type of extraction has been applied for various sample species and matrixes in Example below.

### Example 8: Extraction Capacity of Different Types of Silica Layer Magnetic Beads for Different Sizes of Nucleic Acids in Biological Matrix

In one example, dsDNA ladders with different lengths of DNA fragments (ranging from 25 to 1,000 bp) were spiked in fetal bovine serum (FBS). Smooth silica and SP silica beads were added and incubated in lysis buffer containing chaotropic agents (guanidinium thiocyanate (GITC) in the example, similar components could be but not limited to guanidinium hydrochloride (GuHCl), Guanidinium Tris(2-carboxyethyl)phosphine (GTC), urea, ammonium thiocyanate, lithium chloride, perchlorate salts (sodium-, lithium, etc.), thiourea, caesium chloride, ammonium chloride, iodide salts (such as sodium-, potassium-), trichloroacetic acid and sodium trichloroacetate, bromide salts (such as potassium bromide), formamide etc.), chelating agents (EDTA in the example, similar ones could be but not limited to diethylenetriaminepentaacetic acid (DTPA), ethylene glycol tetraacetic acid (EGTA), nitrilotriacetic acid (NTA), citric acid, lipoic acid, 1,2-bis(o-aminophenoxy) ethane-N,N,N′,N′-tetraacetic acid (BAPTA), ethylenediamine-N,N′-bis(2-hydroxyphenylacetic acid) (EDDHA), N,N′-Bis(2-hydroxybenzyl)ethylenediamine-N,N′-diacetic acid (HBED), triethylenetetramine (TETA), dimercaptosuccinic acid (DMSA), 2,3-dimercaptopropanesulfonic acid (DMPS), thiamine tetrahydrofurfuryl disulfide (TTFD), tris(2-aminoethyl)amine (TAEA) etc.), surfactant (Triton-X in the example, other ones could be but not limited to Tween series, SDS/Sarkosyl (SLS), CTAB, N-Lauroylsarcosine sodium salt, 3-[(3-Cholamidopropyl)-dimethylammonio]-1-propane sulfonate, octyl glucoside, octylphenoxy poly(ethyleneoxy)ethanol, nonyl phenoxypolyethoxylethanol, deoxycholic acid, ammonium lauryl sulfate etc.) and reducing agent (DTT in the example, others can be but not limited to 2-Mercaptoethanol (β-Mercaptoethanol), TCEP (Tris(2-carboxyethyl) phosphine), sodium bisulfite, cysteine, glutathione (GSH), thiourea, tris(2-aminoethyl)amine (TAEA) etc.) and alcohol such as iso-propanol, ethanol, methanol, butanol, pentanol, hexanol etc. Other components, which were not used in the example, can too be added into the lysis buffer. Additional anti-foam agent (such as but not limited to silicones like polydimethylsiloxane (PDMS)/dimethicone, polypropylene glycol, 4-nitroaniline, fatty or stearyl alcohols like tridecanol and octadecan-1-ol, isoamyl alcohol, glycerides, polypropylene-based polyether dispersion, etc.; alone used or mixed) can also be applied into lysis buffer. Proteolytic enzymes/proteases like but not limited to proteinase K, trypsin, pepsin, pronase, subtilisin, papain, chymotrypsin, thermolysin, alcalase, collagenase, ficin etc. can be used to pre-treat the biopsy. RNase treatment can be added to the DNA extraction process to ensure only DNA is extracted. DNase treatment can be done when pure RNA shall be recovered. Carrier RNA poly (A) can be used to improve the extraction of low amount of DNA or RNA in the biological samples. The volume ratio of alcohol:lysis buffer:matrix (FBS) was 1:1:1 in the example. This volume ratio can be changed to shift the main size-range of the extracted nucleic acid, i.e. more alcohol used leads to higher recovery yield of shorter fragments by causing more loss of the longer fragments. Less alcohol used leads to more recovered longer fragments by missing the shorter ones.

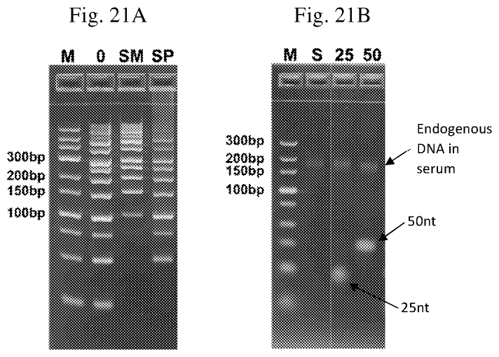

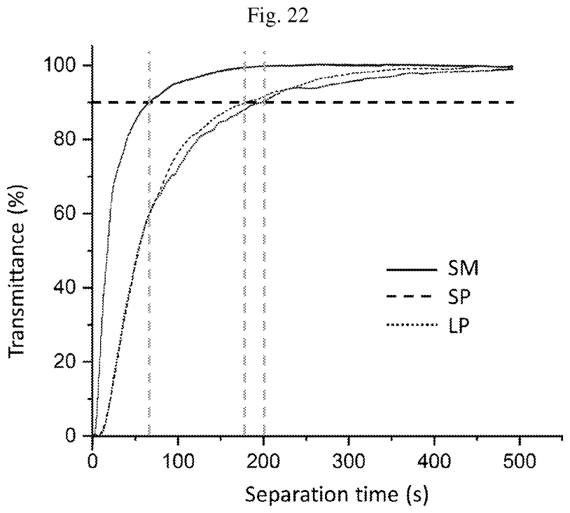

The size profile of eluted DNA of above example is presented by a 4% agarose gel-electrophoresis shown in FIG. 21A. DNA recovered by SM beads is shown in well “SM” and that by SP in well “SP”. Both types of silica beads show good extraction for medium size DNA (100-500 bp). But only SP mesoporous beads show good performance for the capture of short DNA fragments <100 bp. Due to the high-temperature heating during the elution, mesoporous beads seemed to have difficulty to recover long fragments >500 bp. The smear on the top of the gel (well “SP”) proved that long fragments >500 bp were extracted by SP yet without their initial conformations. However, this problem can be solved by proceeding the elution step in thermocycler device with controlled cooling down after heating, which will lead to a better controlled annealing of the long fragments and reduce their degradation.

In another example, DNA extraction in FBS with and without spike-in of short single-stranded oligonucleotides (25 nt or 50 nt) was performed using LP beads. The gel-electrophoresis images (FIG. 21B) expressed a successful recovery of endogenous DNA in FBS (often refers to as cfDNA in serum, with main peak size at around 167 bp) (S), endogenous DNA with spike-in 25 nt oligos (25) and endogenous DNA with spike-in of 50 nt oligos (50), respectively. Here, Ultra Low Range DNA Ladders (10-300 bp) were used as Marker in the gel. This result reveal that mesoporous beads can be used to extract cfDNA from serum. In addition, mesoporous beads are also capable to capture very short single-stranded DNA fragments. Promisingly, this result might be able to be extended to various types of liquid biopsies or to other types of nucleic acids (such as RNA, including miRNA and siRNA).

### Example 9: Rapid Separation of Smooth and Mesoporous Silica Beads

In one example, SM, SP and LP silica beads was evaluated. To do that, SM, SP and LP silica beads were diluted in DI-water to a concentration of 1 mg/mL before being homogeneously dispersed in transparent cuvettes. The separation process is monitored by measuring the transmittance of a green laser (550 nm wavelength) passing through the centre of a transparent cuvette during magnetic separation. To start the measurement, the cuvettes were inserted into the separation position, which is aligned with the laser and sensor. The magnets attract the magnetic beads to the sidewall of the cuvette and the transmittance of the laser in the centre of cuvette increases. The separation speed for each type of beads was shown in FIG. 22. The separation curves expressed that all three types of silica coated beads fulfilled the characteristics of rapid separation, i.e. a near to full separation within 10 min. In ore details, the separation of SM beads reached to over 90% within one minute and was 100% finished within three minutes. The separation of SP and LP mesoporous beads is slower compared to the SM beads, with 90% separations in about three minutes, and 100% completion of separation in less than 10 minutes.

This example highlights that, despite the multiple layers of silica on their surface, SP and LP beads still exhibit rapid magnetic separation speeds.

### Example 10: Removal of the Soft Template of Surfactant Generated During the Mesoporous Coating

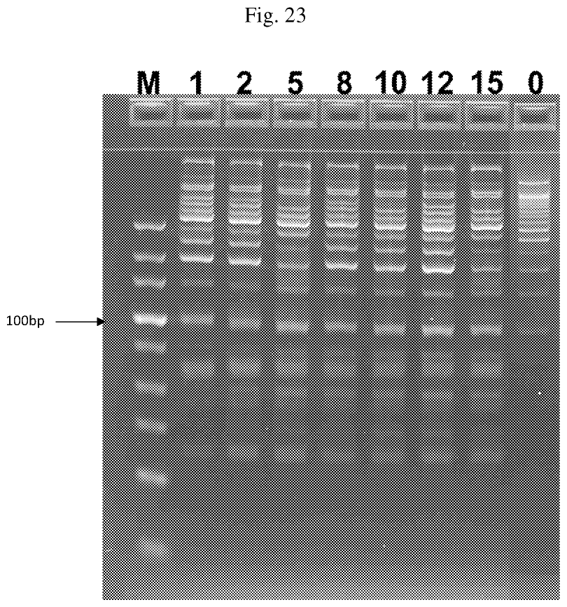

The removal of the soft template of the surfactant generated during the mesoporous coating is crucial for multiple applications, including for nucleic acid extractions (by allowing nucleic acids to enter the mesopores). Indeed, when some surfactants remain in the pores, short nucleic acids cannot be extracted, as seen for no short DNA fragments (<100 bp) observed in the gel image shown in FIG. 23 (0 for no treatment).

In one example, mesoporous silica beads coated using CTAB as surfactant were incubated in 10% hydrochloride acid (in ethanol) for 30 min at room temperature. Thereafter, DNA extraction was performed using same procedure as described in previous examples. As seen in the gel electrophoresis, short DNA fragments (<100 bp) were successfully extracted indicating the free access of the mesopores corresponding to the removal of the CTAB.

In another example, mesoporous silica beads coated using CTAB as surfactant were incubated in 10% hydrochloride acid (in ethanol) in an ultrasonic bath for 30 min at room temperature. Also here, short DNA fragments were successfully extracted.

In another example, mesoporous silica beads coated using CTAB as surfactant were treated with 10% hydrochloride acid (in ethanol) for 21 hours at room temperature. Also here, short DNA fragments were successfully extracted, suggesting that CTAB template was removed. No apparent iron leakage was observed (iron leakage=clear yellow supernatant by visual observation).

In another example, mesoporous silica beads coated using CTAB were treated with 10% hydrochloride acid (in ethanol) for 30 min at room temperature. Here, a clear yellow supernatant was observed indicating the leakage of the iron cores of the beads. This suggests that the mesoporous silica layer did not resist those harsh conditions. However, this method of surfactant removal might work when the coated silica layer is thicker.

In another example, mesoporous silica beads coated using CTAB were treated with hydrogen peroxide (H₂O₂) for time ranging from 1 min to 15 min at room temperature. All treated beads showed the capability of capturing short DNA fragments (FIG. 23) indicating successful removal of the surfactant templates. In addition, no iron leaking was observed by visual inspection of the colour of the supernatant.

### Example 11: Size Selective Extraction of Small DNA Fragments (≤300 bp) in FBS with Spiked-In dsDNA Ladders (25-1,000 bp) with Quantitative Analysis

In DNA LoBind tubes (Eppendorf), 250 μL of FBS was incubated with 250 μL of lysis buffer (containing 6M GITC and 50 μL of IPA) for 10 minutes at room temperature. After that, 1 μg of DNA ladders (consisting of 50% of GeneRuler Low Range DNA Ladder (size ranging from 25 bp to 700 bp) and of 50% of 50 bp DNA ladder (size ranging from 50 bp to 1,000 bp) (ThermoFisher)) were spiked-in the FBS/lysis buffer solution. After addition of 1 mg of mesoporous SP silica beads, the solution was incubated for 10 minutes at room temperature with agitation. During this step, the larger DNA fragments are expected to bind to the smooth beads. After magnetic separation, the supernatants, containing only the smaller DNA fragments, were transferred to a new DNA LoBind tube with an additional 325 μL of isopropanol (making total used IPA in the second extraction of 375 μL) and 500 μg of fresh mesoporous SP silica beads. Following a 10-minute incubation step at room temperature with agitation, the beads were washed twice with a solution containing 3M GITC-ethanol (containing 50% of 3M GITC-EDTA mixture and 50% of EtOH), before an additional three washes performed with 80% ethanol (all washing are performed using magnetic separation, and at room temperature). Finally, after completion of the last wash, the supernatant is removed and 50 μL of elution buffer (10 mM Tris buffer, pH 8.9) is added to the beads. After incubation at 90° C. for 1 h followed by a slow cooling down, the tube is placed on a magnetic rack and the supernatant, containing the eluted DNA, is transferred to new DNA LoBind tubes. The eluted DNA samples were stored in −20° C. freezer until analysis on a 4% agarose gel (E-Gel® EX, Invitrogen).

In comparison, DNA extraction without size-selection was also performed with SP beads. Here, DNA ladders (1 μg in total, containing 25-1000 bp fragments) were spiked in 250 μL FBS. 250 μL of lysis buffer and 375 μL IPA was mixed to the sample. After an incubation of 10 min and washing steps (twice with a solution containing 50% of 3M GITC-EDTA and 50% of EtOH), and three times with 80% ethanol), the recovered DNA was eluted into 50 μL elution buffer. The eluted DNA was stored at −20° C. before analysis on a 4% agarose gel.

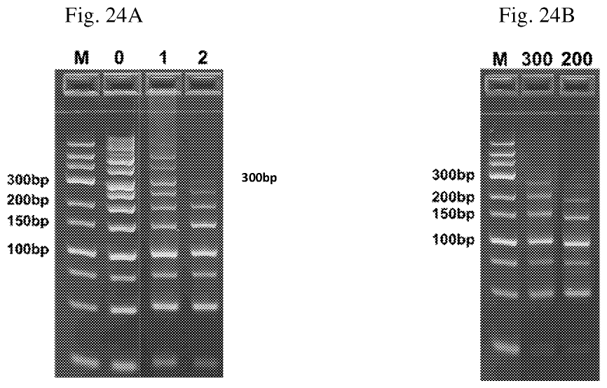

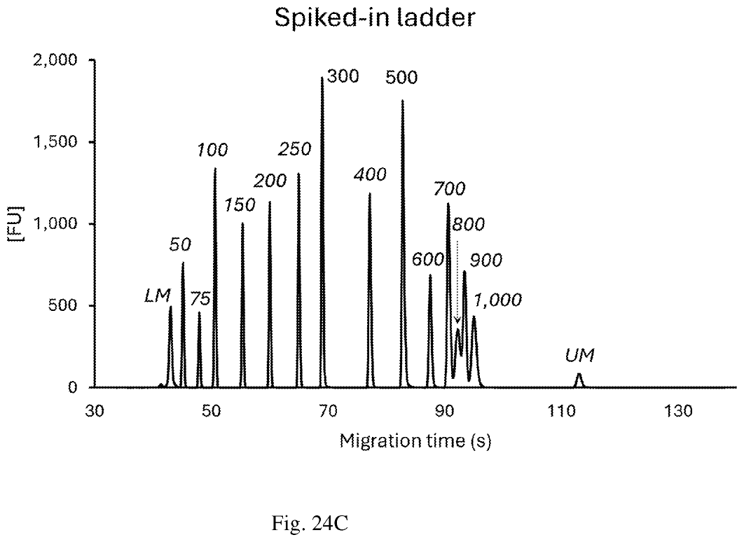

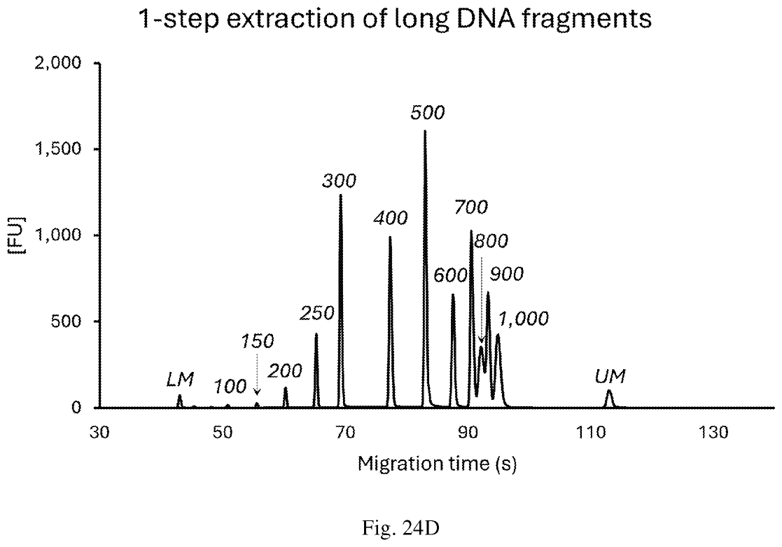

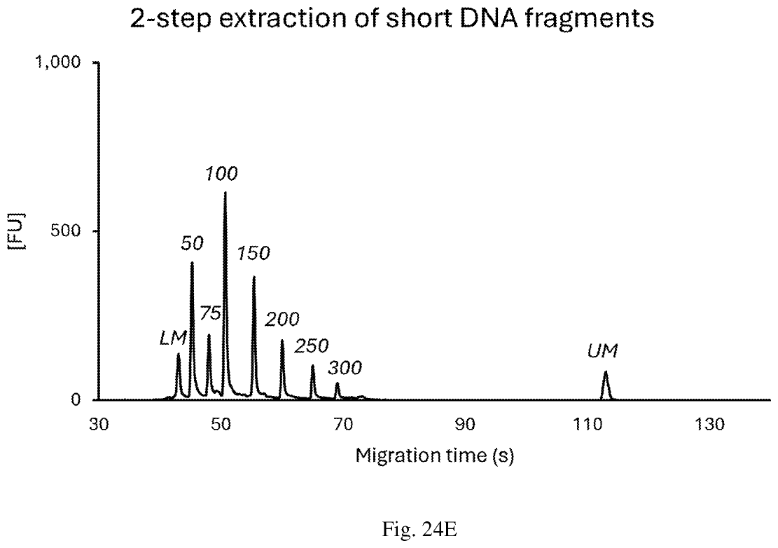

FIG. 24A shows a gel image of recovered DNA via this 2-step extraction. GeneRuler Low Range Ladders (25-700 bp) were used as Marker (labelled as “M”). Well 0 shows the size profile of the spike-in DNA sized from 25 to 1,000 bp and well 1 shows the bands of recovered DNA without the size-selective extraction. Well 2 expresses the recovered DNA after the size-selective extraction. The results clearly express that all long fragments over 300 bp were removed through the first extraction step using smooth silica beads whilst the short ones were successfully recovered by mesoporous SP beads. In addition, the threshold of DNA size-selection can be well controlled by varying the ratio between lysis buffer and isopropanol. In one example, 80 μL IPA was added to the lysis buffer at the first extraction step yet keeping the ratio between lysis buffer and IPA for the second extraction, the threshold of size-selection was lowered from 300 bp to 200 bp, as shown in FIG. 24B.

For a more precise estimation of the size selectivity and of the amount of DNA recovered, Bioanalyzer High Sensitivity DNA chip (Agilent) was used. To prepare the DNA samples, the following procedures were used. In DNA LoBind tubes, 250 μL of FBS was incubated with 250 μL of lysis buffer (containing 5M GITC and 50 μL of IPA) for 10 minutes at room temperature. After that, 1 μg of DNA ladders (consisting of 50% of GeneRuler Low Range DNA Ladder and of 50% of 50 bp DNA ladder were spiked-in the FBS/lysis buffer solution. After addition of 1 mg of smooth (SM) silica beads, the solution was incubated for 10 minutes at room temperature with agitation. During this step, the larger DNA fragments are expected to bind to the SM beads.

After magnetic separation, the SM beads (containing the longer DNA fragments) and the supernatant (containing the smaller DNA fragments) were separated.

For selection of larger DNA fragments (1-step procedure), the SM beads were washed twice with a solution containing 3M GITC-ethanol, before an additional three washes with 80% ethanol. Finally, after magnetic separation, the supernatants were removed and 50 μL of elution buffer (10 mM Tris buffer, pH 8.9) was added to the beads. After incubation at room temperature for 15 minutes, the tubes were placed on a magnetic rack and the supernatant, containing the eluted DNA, was transferred to new DNA LoBind tubes. The eluted DNA samples were stored in −20° C. freezer until analysis on the Bioanalyzer using High Sensitivity DNA chip.

For selection of the smaller DNA fragments (2-step procedure), the supernatants were transferred to new DNA LoBind tubes with an additional 325 μL of isopropanol and 500 μg of mesoporous SP silica beads. Following a 10-minute incubation step at room temperature with agitation, the beads were washed twice with a solution containing 3M GITC-ethanol, before an additional three washes with 80% ethanol. Finally, after magnetic separation, the supernatants were removed and 50 μL of elution buffer was added to the beads. After incubation at 90° C. for 1h, followed by a slow cooling down, the tubes were placed on a magnetic rack and the supernatant, containing the eluted DNA, was transferred to new DNA LoBind tubes. The eluted DNA samples were stored in −20° C. freezer until analysis on the Bioanalyzer using High Sensitivity DNA chip.

The results, shown in FIGS. 24C-C represent the amount and size distribution of the analysed DNA, with in FIG. 24C the initial spiked-in ladder (25-1,000 bp) in elution buffer at 20 ng/μL (mimicking sample a 100% recovery for all DNA fragments), in FIG. 24D the DNA recovered using the 1-step procedure with SM beads (selection of the longer DNA fragments) and in FIG. 24E the DNA recovered using the 2-step procedure with SM beads first, then SP beads (selection of the smaller DNA fragments). The number above each peak corresponds to the theorical size in base pairs for the DNA in each peak (in a Bioanalyzer chip, DNA fragment migration time generally correlates with fragment size, with larger fragments migrating more slowly). The graphical representations show that the extractions conditions were able to successfully size selectively extract only the longer fragment DNA (FIG. 24D) or only the shorter fragments (FIG. 24E). In complement, the percentage of recovery for specific size range of DNA was estimated using the concentration of DNA estimated by the bioanalyzer software (2100 Expert Software, Agilent) and by comparison with control sample (initial spiked-in ladder at 20 ng/μL). The 1-step extraction was able to extract on average 80% of the DNA fragments between 320-1,300 bp (confirming results in FIG. 24D), whilst the 2-step extraction was able to extract on average 70% of the DNA fragments between 40-170 bp and 49% of the DNA fragments between 40-320 bp (confirming results in FIG. 24E). Therefore, taken together, these results show that our procedure is able to size selectively extract DNA fragments from a complex matrix such as serum (FBS). And, with optimization of the bead coating and of the reaction conditions (such as with a pre-treatment step with proteinase K, a change in the ratio of GITC and isopropanol, an improvement in the elution conditions . . . ), the percentage of recovery of the smaller DNA fragments can be significantly improved. Of specific interest, we believe that this procedure could be the foundation for a kit for the size selective extraction of small DNA fragments from liquid biopsies (including plasma and serum). The exact threshold for the size selective extraction could be adjusted (by for example changing the ratio between lysis buffer and isopropanol) to be either below 150 bp and/or below 300 bp depending on the use. This size range is of particular interest as it corresponds to the size of most ctDNA (smaller than cfDNA of non-tumour origin), and that the size selective extraction of smaller DNA from liquid biopsies could increase the ratio of ctDNA (compared to cfDNA and genomic DNA), which in turn could improve diagnostic, monitoring and treatment options for cancer patients by improving analysis of ctDNA in key methods (including digital PCR, RT-PCR, NGS . . . ).

### Example 12: Elution Methods after Nucleic Acid Extraction with Mesoporous Beads

In one example, DNA captured by mesoporous beads was eluted in 10 mM Tris buffer at 30° C., 40° C., 55° C., 60° C., 70° C. and 90° C. for 1 hour, respectively. DNA showed the highest recovery rate without DNA-degradation when the elution step was proceeded at 90° C.

In another example, DNA captured by mesoporous beads was eluted in 10 mM Tris buffer at 90° C. for 5, 10, 15, 20, 30, 40, 45, 50, 55 and 60 minutes. The results show that a complete elution was achieved after the beads were heated for 50 minutes.

In another example, DNA captured by mesoporous beads was eluted in 10 mM Tris buffer with pH 7, 8 and 8.9, respectively. The results show that a higher pH (=8.9) of the elution buffer improved the recovery of the extracted DNA. The pH value of the elution buffer can vary between 7 and 10. Elution buffer with pH larger than 10 might cause the degradation of extracted DNA.

### Example 13: Size Selective Extraction of Spiked-In dsDNA Ladders (25-1,000 bp) in Buffer Using PEG and Carboxyl-Coated LS Magnetic Beads

In this example, we wanted to test the possibility for using porous silica layer magnetic beads in procedures refer to as solid-phase reversible immobilization (SPRI), in which carboxyl magnetic beads in extraction buffer containing specific concentration of PEG and salt are used to allow for DNA size selections.

Beads preparation: Porous LS layer magnetic beads were made as per described in the example 5. Amine groups from the APTES were then covalently bound to the silanol groups of the silica surface before further modification by binding of carboxyl groups to the amine groups for the creation of carboxyl-coated LS magnetic beads. Those beads were then resuspended at 2 mg/mL in either extraction buffer A (containing 20% PEG-8000 and 2M NaCl in 10 mM Tris-HCl buffer, pH 8.9) or in extraction buffer B (containing 30% PEG-8000 and 3M NaCl in 10 mM Tris-HCl buffer, pH 8.9).

First step of procedure: In DNA LoBind tubes (Eppendorf), 1 μg of DNA ladders (consisting of 50% of GeneRuler Low Range DNA Ladder (size ranging from 25 bp to 700 bp) and of 50% of 50 bp DNA ladder (size ranging from 50 bp to 1,000 bp) (ThermoFisher)) were spiked-in 250 μL of 10 mM Tris-HCl buffer, pH 8.9 (classic buffer used as elution buffer in DNA extraction procedures). After addition of 500 μg of carboxyl-coated LS magnetic beads in 250 μL of extraction buffer A, the solution was incubated for 10 minutes at room temperature with agitation. During this step, the larger DNA fragments are expected to bind to the beads. After magnetic separation, the beads were used for size selection of longer DNA fragments (see Option A) and the supernatant was used for a second step of extraction for size selection of smaller DNA fragments (see Option B).

Option A for size-selection of longer DNA: After magnetic separation and removal of the supernatant, the beads were washed three times with 80% ethanol. After the last wash, the supernatant was removed by magnetic separation and 50 μL of elution buffer (10 mM Tris-HCl buffer, pH 8.9) was added to the beads. After incubation at 50° C. for 1 h with agitation, followed by a slow cooling down, the tube was placed on a magnetic rack and the supernatant, containing the eluted DNA, was transferred to a new DNA LoBind tube. The eluted DNA was analysed in 4% agarose gel and stored at −20° C.

Option B for size-selection of smaller DNA: After magnetic separation, the supernatant, containing only the smaller DNA fragments, was transferred to a new DNA LoBind tube. The supernatant was supplemented with 1,000 μg of carboxyl-coated LS magnetic beads in 500 μL of extraction buffer B. After an incubation of 10 minutes at room temperature with agitation, the beads were washed three times with 80% ethanol. After the last wash, the supernatant was removed by magnetic separation and 50 μL of elution buffer was added to the beads. After incubation at 50° C. for 1 h with agitation, followed by a slow cooling down, the tube was placed on a magnetic rack and the supernatant, containing the eluted DNA, was transferred to a new DNA LoBind tube. The eluted DNA was analysed in 4% agarose gel and stored at −20° C.

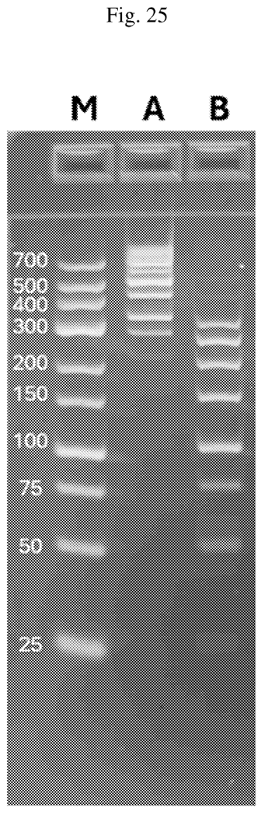

FIG. 25 shows a gel image of the recovered DNA for both options. The well labelled “M” represents the size marker (GeneRuler Low Range DNA Ladder, with size ranging from 25 bp to 700 bp), and the white number corresponds to the size of each fragments of the marker in base pairs (bp). The well “A” shows the size profile of the recovered DNA with the size-selective extraction for longer DNA fragments (Option A), whilst the well “B” shows the size profile of the recovered DNA with the size-selective extraction for smaller DNA fragments (Option B).

The gel shows that the carboxyl-coated LS magnetic beads can successfully size select DNA depending on the composition of the extraction buffer. In this example, the 1-step procedure (option A) allows for the specific extraction of DNA fragments of 300 bp or longer. At the contrary the 2-step procedure (option B) allows for the extraction of DNA fragments of smaller than 300 bp. Interestingly, the threshold for the DNA size-selection can be well controlled by varying the mass/volume of beads/extraction buffer used, as well as by changing the amount of PEG and NaCl in extraction buffer. Moreover, this procedure can be scaled for lower to higher volume of sample, and automation might be implemented to facilitate every type of usage.

Overall, this example highlights the capacity of using carboxyl-coated LS magnetic beads in specific extraction buffers (containing NaCl and PEG) for size selection of smaller or longer dsDNA. This demonstrates the potential of carboxyl-coated porous silica magnetic beads for a wide range of activity requiring specific size of DNA, such as for the clean-up and precise size selection needed in NGS library preparation.

What is claimed is:

1. A method for producing one or more uniform spherical nano/microsized magnetic beads, comprising:
1) providing magnetic nanoparticles with size between about 1 nm to about 100 nm and assembled magnetic beads sized between about 50 nm to about 5 microns;
2) hydrophobically coating the nanoparticles dispensed in a highly volatile organic solvent that is immiscible with aqueous solution, wherein the aqueous solution comprises a thickening agent with adjustable viscosity and a surfactant;
3) producing an emulsion in a pre-mixer by dispersing flow of the hydrophobic solvent comprising the nanoparticles in a continuous flow of aqueous solution that is immiscible with the solvent;
4) producing emulsion in a pre-mixer by dispersing flow of aqueous solution in a continuous hydrophobic solvent flow comprising nanoparticles;
5) dispersing emulsion from pre-mixer to a rotating shear device, thereby providing uniform emulsion, temperature control, and viscosity control;
6) adjusting emulsion droplet size by adjusting the temperature and viscosity of emulsion temperature; and
7) evaporating solvent in emulsion droplet to form assembled micrometer or submicrometer sized bead.

2. The method of claim 1, wherein the highly volatile organic solvent is selected from cyclohexane, pentane, or toluene.

3. The method of claim 1, wherein the thickening agent with adjustable viscosity is selected from Polyvinylpyrrolidone (PVP), dextran, Polyvinyl alcohol (PVA), Polyethylene glycol (PEG), and Gelatin.

4. The method of claim 1, wherein evaporating solvent occurs by heating evaporation or spray evaporation.

5. The method of claim 1, wherein one or more magnetic beads have uniform diameter and spherical shape.

6. The method of claim 1, wherein the magnetic beads have a uniform diameter ranging from about 50 nm to about 5 microns.

7. The method of claim 1, wherein the magnetic beads have a large mass of magnetic material, wherein the magnetic material comprises at least about 50% of total mass of the bead.

8. A method for coating a layer of continuous silica over one or more magnetic beads, comprising:
1) optionally producing one or more silicate layers using ion-exchanged sodium silicate in aqueous solution;
2) separating magnetic beads using different magnetic strength during the silica coating process; keeping silicate coated magnetic beads in alkalic solution before next step;
3) producing one or more silane layers based on the first silicate;
4) producing a silane layer-coating, with optional sonication
5) providing different roughness of the above mentioned silane surface resulted by different coating conditions;
6) producing a third corrugated silane layer in the mix of aqueous solution and immiscible (with aqueous solution) organic solvent (such as cyclohexane, toluene, pentane, chloroform);
7) providing morphologies of coated silica mesoporous surface resulted by different reaction conditions regarding heating temperatures, sonication powers, types of co-surfactant with different concentrations, types of catalysts, etc.; and
8) providing difference of the bead-size/thickness of silica layer resulted by different coating conditions.

9. The method of claim 8, wherein said bead is microporous, mesoporous, or macroporous.

10. A kit comprising microporous, mesoporous, or macroporous silica beads with specifically designed pore sizes, the appropriate buffers (adsorption, rinse, and desorption) and instructions for use.

11. A method for affinity separation, comprising using any composition and/or methodology disclosed herein.

12. A method for ion-exchange separation, comprising using any composition and/or methodology disclosed herein.

13. A method for bio-separation, comprising using any compositions and/or methodology disclosed herein.

14. Use of magnetic porous beads for controlling rate of a reaction, wherein the pore size controls the reaction.

15. Use of the magnetic porous beads of claim 14, wherein the pores comprise an immobilized enzyme and reactive center.

16. Use of a mesoporous material for controlling rate of a reaction.

17. A mesoporous magnetic bead with a silica layer.

18. A kit for extracting nucleic acids, wherein said kit comprises a mesoporous magnetic bead with a silica layer, wherein said bead has stronger binding capacities for nucleic acids than flat silica surfaces.

19. The kit of claim 18, wherein said nucleic acids comprise ssDNA, dsDNA, ssRNA, and dsRNA.

20. A composition for extracting short single-stranded oligonucleotides, comprising a silica coated magnetic bead with pores of suitable diameter and depth (LP pores).

21. A method for separating small nucleic acid fragments from a complex matrix, comprising using a porous magnetic bead with a silica layer to bind the nucleic acid fragments.

22. The method of claim 21, wherein said nucleic acid fragments are ctDNA having a length of less than about 300 bp.

23. The method of claim 21, wherein the complex matrix comprises plasma or other biological fluids listed in the application.

24. A method for size-selective extraction of DNA, comprising using one or more carboxyl-coated mesoporous magnetic beads to bind the DNA.

25. The method of claim 24, wherein the DNA ranges from about 25 bp to about 300 bp.

26. The method of claims 24-25, wherein the DNA size-selection can be well controlled by varying the mass/volume of beads/extraction buffer, and/or varying the among of PEG and NaCl in the extraction buffer, and/or varying the buffer content (such as a GITC based extraction), and/or bead type, and/or solvent (such as an alcohol, like methanol, ethanol, propanol, isopropanol, n-, sec-, or iso-butanol, etc.).

27. A carboxyl-coated porous silica magnetic bead.

28. A method for extracting cfDNA from biological fluid samples, comprising using one or more porous and/or mesoporous silica-coated beads to bind cfDNA.

29. A method for extraction of nucleic acids from liquid biopsy, comprising using one or more porous and/or mesoporous silica-coated beads to separate cfDNA from biological fluid samples.

30. A kit for extraction of nucleic acids, comprising one or more porous silica-coated beads.

31. A method for increasing specific surface area of mesoporous silica-coated beads, comprising controlling pore size and pore structure of the silica layers.

32. The method of claim 31, wherein beads having large pore (LP) and small pore (SP) silica layers both have significantly higher specific surface area compared to that of beads coated with smooth silica layer (SM).

33. The method of claims 31-32, wherein the specific surface area increases by one or more orders of magnitude.

34. A mesoporous silica-coated bead, wherein said bead comprises a surface functional group selected from amine, carboxyl, azide, proteins streptavidin, antibodies, enzymes, peptides, oligo-dT, nucleic acids, and crown ethers.

35. A method for surface coating multi-layer silica on spherical magnetic beads, comprising stepwise coating three different silica layers: a) producing a surrounding silicate layers around the iron oxide core; and b) producing corrugated/mesoporous silica layers that have high surface-to-volume ratio.

36. The method of claim 35 or any preceding claim, wherein parameters for coating each layer can be adjusted to create customized beads with different silica layer size, different types of pores, different size/depth of the pores.

37. The method of claims 35-36 or any preceding claim, wherein the outer silica layer can be further functionalized with specific reactive groups or coated with a range of biochemical compounds to facilitate usage in specific applications.

38. A method for extracting a molecule from solution, comprising using a surface-coated multi-layer silica spherical magnetic bead, wherein said bead is coated with a functional group that interacts with said molecule and said molecule is smaller than the size of the pore of the bead.

39. The method of claim 36, wherein said biochemical compounds are selected from peptides, proteins, and antibodies.

40. A composition comprising SAv-modified LP beads.

41. A composition comprising SAv-modified mesoporous beads with pore sizes of specific size.

42. A method for separating biotin from solution, comprising using SAv-modified LP beads or SAv-modified LS beads.

43. Any methodology, use, device, composition, or kit disclosed herein.
